(19) United States
(12) Patent Application Publication
(10) Pub. No.: US 2006/0241495 A1
(43) Pub. Date: Oct. 26, 2006

(54) Wound healing monitoring and treatment

(75) Inventor: Andrew F. Kurtz, Macedon, NY (US)

Correspondence Address: Mark G. Bocchetti;Patent Leagl Staff, Eastman Kodak Company, 343 State Street, Rochester, NY, 14650-2201, US

(73) Assignee: Eastman Kodak Company

(21) Appl. No.: 11/087,183

(22) Filed: Mar. 23, 2005

(51) Int. Cl. A61B 6/00

(52) U.S. Cl. 600/476

(57) ABSTRACT

A polarization based diagnostic device (200) for optically examining the medical condition of tissue (290) comprises an illumination optical system (205), comprising a light source (220) and beam shaping optics. An optical detection system (210) comprises imaging optics and an optical detector array, which detects light from the tissue. Polarizing optics provided in both the illumination optical system and the optical detection system are crossed and pass orthogonal polarization states. Iterative rotational means rotate the orthogonal polarization states relative to the tissue being examined. Image enhancement means includes image processing, sequential multi-spectral illumination and imaging, and image focus control to facilitate quality imaging at varying depths within the tissue. A controller (215) operates the light source, the detector array, the multi-spectral illumination and imaging, image focus control, and image processing.

### FIELD OF THE INVENTION

The invention relates generally to a monitoring device for examining the state of tissues, and in particular a device that examines the condition of collagen structures within tissue using a light based diagnostic device in close proximity with the skin of a patient. The device of the invention is optimally used in cooperation with a light therapy device.

### BACKGROUND OF THE INVENTION

In general, the healing of wounds, burns, and other injuries is an uncertain endeavor. The clinician cannot be certain about the condition of the tissue being treated, the efficacy of treatments, and whether further treatments or a change in treatments is appropriate. As a particular example, many chronic wounds, such as pressure ulcers or venous stasis ulcers linger for months or even years, often despite the various treatments being applied. These wounds are particularly intractable for a variety of reasons, with age, nutrition, diabetes, infection, marginalized immune systems, and other factors; all contributing to the ongoing difficulties in healing. In most cases, such wounds are chronic because the wound healing is stalled relative to one or more aspects of the process. In such circumstances, it is not unusual for the clinician to be unsure about the status of the wounded tissue, at what point in wound healing the tissue is held up, and what new treatment modality should be applied.

There are a variety of methods and devices that can be utilized to aid ongoing diagnosis. For example, tissue biopsies can be taken, and used in tissue cultures and histology. These traditional methods are disadvantaged by the time delay in evaluating tissue cultures or histology, which easily can be a week or two. Additionally, these approaches are invasive, and actually cause further damage to the tissue. As histology relies on thin slices of tissue, which are dyed and examined optically with a microscope, histology only provides a direct indication of the tissue structure in two-dimensions.

Alternate technologies have been developed for non-invasive histology or tissue imaging, including X-ray, magnetic resonance imaging (MRI), computed axial tomography (CAT) scanning, and positron emission tomography (PET). These technologies are used for a variety of applications (mammography, brain scans, etc.), but are seldom used for examining soft tissue wounds, and can expose the patient to high energy radiation (x-rays, etc.). Ultrasound, which is widely used for pre-natal examination, can also be used for examining wounds. In particular, Longport Inc. (Glen Mills, Pa.) offers a high frequency (20 MHz) ultrasound scanner, described in U.S. Pat. No. 6,073,045 (Dyson et al.) that scans tissue to modest depths (2 cm) but with “high” spatial resolution (65 microns).

However, because many biological structures, including cells, are much smaller than 65 microns, there is a need for other imaging technologies that offer higher resolution, but at a lower cost than MRI and some other medical imaging technologies. There are a variety of technologies, including confocal scanning microscopy, optical coherence tomography (OCT), second harmonic generation (SHG) based microscopy, which apply optical techniques to obtain high resolution images either in-vivo or in-vitro. In these cases, imaging resolution can be as little as a few microns, which certainly enables detection of much finer structures than does ultrasound. On the other hand, light absorption and scatter limit the imaging depth in most tissues to only ˜3-4 mm. While many of these systems have been used for optical histology, providing a two-dimensional image of the tissue, newer technologies, such as full field OCT systems, enable three-dimensional images.

As wounds heal, they normally progress through a sequence of overlapping interactive phases, starting with coagulation and progressing through inflammation, proliferation (which includes granulation, angiogensis, and epithelialization), and remodeling. Success in wound healing is very much dependent on the rebuilding of the extra-cellular matrix (ECM), which is initially dependent on fibroblasts. Fibroblasts migrate into the wound site, and begin to build the ECM by depositing a protein called fibronectin. The fibronectin is deposited with some directionality, mirroring the axis of the fibroblasts. The fibroblasts then produce collagen, with the collagen deposition generally aligned to the fibronectin pattern. Over time, fibronectin is replaced by Type III collagen and ultimately by Type I collagen. As the wound contracts, and is subsequently remodeled and influenced by stresses from neighboring tissues, the collagen becomes increasingly organized. Even late in the remodeling phase, which can end six months to a year post injury, collagen in a scar will be replaced and rearranged as the wound attempts to regain its original function. As collagen is optically birefringent, the wound healing processes involving the formation and remodeling of the extra-cellular matrix (ECM) could potentially be observed as an indication of the status and viability of the ongoing wound healing. This information could in turn be used to guide the clinician in the application of further wound therapies, including light therapy.

Several imaging technologies, including OCT systems, SHG systems, and the Longport ultrasound scanner have been used for non-invasive histology, including the examination of wounds. Furthermore, some of these systems have used polarization sensitive optical systems, so that optically birefringent tissue structures can be examined. Some of these systems have been used to detect birefringent collagen tissue structures, such as are present in tendons, ligaments, and healed scars. There are several such examples.

As a first example, polarization sensitive microscopes are well known in the art, and are commonly used in biomedical laboratory work. Exemplary prior art systems are described in U.S. Pat. No. 5,559,630 (Ho et al.) and U.S. Pat. No. 5,835,262 (Iketaki et al.). Metripol (Oxford, United Kingdom), offers the Metripol Birefringence Imaging System as upgrade to conventional polarization microscopes, using a rotating polarizer, a CCD camera, and custom software, to enhance the polarization sensitivity. This system can be used for biological applications to examine cell division and tissue strain, as well as industrial applications to examine strain and defects in polymers, glass, and silicon wafers. While polarization microscopes work well for examining tissue, such systems are both large and expensive, and require tissue or cellular samples that can be examined in-vitro (on a slide) rather than in-vivo.

As previously mentioned, optical coherence tomography (OCT) systems have been used to provide non-invasive optical histology of tissue in-vivo. An OCT system is basically a fiber optic based interferometer, typically using a low coherence (broad band, for example ˜30-70 nm) light source. The system is provided with a sampling arm, which includes a fiber optic probe to direct light onto the tissue. The system also has a reference fiber optic arm with a retro-reflector. The interference effect allows OCT systems to control the depth of focus, so that a small longitudinal distance is in focus. Images are constructed by first measuring the in-depth profile of the backscattered light intensity in the axial (depth) direction. In-depth profiling is performed by measuring the echo time delay and intensity of backscattered or reflected light. Distance or spatial information is determined from the time delay of reflected echoes. To create a two-dimensional image, the fiber optic beam is moved laterally across the surface (x-axis) and in-depth profiles (z-axis) are obtained at discrete points along the surface. The net result is that the resolution (4-20 microns) and dynamic range of the sample are in focus and enhanced as compared to the portion of the sample the pre-focused beam traveled through. This can be particularly advantageous for imaging in turbid, light scattering optical media, such as tissue. Exemplary OCT system patents include U.S. Pat. Nos. 5,659,392 and 6,034,774 (both to Marcus et al.), both of which are assigned to the same assignee as the present invention. As one example, Imalux Corp. (Cleveland, Ohio) offers the NIRIS Imaging System, with a super luminescent diode light source and 10-20 micron resolution, for $65,000.

Polarization sensitive OCT systems have also been developed. An exemplary prior art system, described in U.S. Pat. No. 6,208,415 (DeBoer et al.), has been used at Massachusetts General Hospital to examine dermal tissues, burns, scars, and tendons. Another exemplary prior art OCT system, described in U.S. Pat. No. 6,615,072 (Izatt et al.) is equipped with a polarization compensation system, so as to desensitize the device to polarization degradation effects that occur in bent single mode optical fibers. Another similar system is a polarization sensitive low coherence reflectometer, such as described in U.S. Pat. No. 5,459,570 (Swanson et al.) which has 11 micron resolution and 120 dB signal to noise ratio. Although the fiber optic OCT systems can have a small probe for in-vivo testing, these systems are complicated and expensive, and not likely to be used by a clinician in wound assessment either in the field or in many clinical settings.

There are other diagnostic devices for optical examination of tissue that could be applicable. For example, polarimeters have been described which use ultrashort (femtosecond) laser pulses to induce biological components, such as collagen, to emit light by second harmonic generation processes. As such systems utilize elaborate laser sources (for example a Ti:sapphire laser pumped by a Nd:YVO laser), these systems will likely also be appropriate for general use.

The need for inexpensive, portable optical devices for tissue diagnosis has also not gone unrecognized in the prior art. For example, Electro-Optical Sciences Inc. (Irvington, N.Y.) is developing the Melafind system for detecting melanomas, which is described in U.S. Pat. Nos. 6,081,612 and 6,307,957 (both to Gutkowics-Krusin et al.). The Melafind probe emits 10 pulses of light, each a different wavelength, and then detects light scattered off of tissue. The Melafind device then uses multi-spectral analysis and a database to diagnose lesions within 2 minutes. While the Melafind device is depicted as a hand-held unit, the Siascope system, which is being developed by Astron Clinica (Cambridge, United Kingdom), provides a hand-held probe tethered to a mobile station, as a melanoma detection and diagnosis system. This system, which is described in U.S. Pat. No. 6,324,417 (Cotton), directs infrared and red light at the suspect tissue, and uses color space information to characterize the tissue.

Some portable optical devices for tissue diagnosis using polarization optics have also been developed. As an example, Lekam Medical (Devon, United Kingdom) offers the Cytoscan, which uses orthogonal polarization spectral imaging technology developed by Cytometrics Inc., and described in a U.S. Pat. No. 5,983,120 (Groner et al.); U.S. Pat. No. 6,438,396; and U.S. Pat. No. 6,650,916 (both to Cook et al.). This system is designed to provide images of the micro-circulatory vascular network, and is not optimized to examine the collagen network present in the dermal layers of skin. The Cytoscan system does not provide the proper optical wavelengths, high contrast polarizers, or polarization control to properly examine the collagen networks.

As stated previously, the ability to examine the collagen networks forming in the extracellular matrix could improve the healing therapies applied to the wounded tissue. While such information might effect the application of antibiotics and other topical agents, collagen welds, and growth factors to impact healing progress, the relationship to the use of light therapy devices is particularly interesting. External light therapy has been shown to be effective in treating various medical conditions, for example, seasonal affective disorder, psoriasis, acne, and hyperbilirubinemia common in newborn infants. Light therapy has also been employed for the treatment of wounds, burns, and other skin surface (or near skin surface) ailments.

In the 1960's and 1970's researchers in Eastern Europe undertook the initial studies that launched modern light therapy. One such pioneer was Endre Mester (Semmelweiss Hospital, Budapest, Hungary), who in 1966, published the first scientific report on the stimulatory effects of non-thermal ruby laser light (694 nm) exposure on the skin of rats. Professor Mester found that a specific range of exposure conditions stimulated cell growth and wound healing, while lesser doses were ineffective and larger doses were inhibitory. In the late 1960's, Professor Mester reported the use laser light to treat non-healing wounds and ulcers in diabetic patients. Mester's 70% success rate in treating these wounds lead to the development of the science of what he called “laser biostimulation.”

Presently, there are over 30 companies world wide that are offering light therapy devices for a variety of treatment applications. These devices vary considerably, with a range of wavelengths, power levels, modulation frequencies, and design features being available. In many instances, the exposure device is a handheld probe, comprising a multitude of light emitters; that can be directed at the patient during treatment. The light emitters, which typically are laser diodes, light emitting diodes (LEDs), or combinations thereof, usually provide light in the red-IR (˜600-1200 nm) spectrum, because the tissue penetration is best at those wavelengths. In general, both laser light and incoherent (LED) light seem to provide therapeutic benefit, although some have suggested that lasers may be more efficacious. Light therapy is covered by a variety of terms, including low-level-laser therapy (LLLT), low-energy-photon therapy (LEPT), and low-intensity-light therapy (LILT). Despite the emphasis on “low”, many of the products marketed today output relatively high power levels, of up to 1-2 optical watts. Companies that presently offer light therapy devices include Thor Laser (United Kingdom), Omega Laser Systems (United Kingdom), MedX Health (Canada), Quantum Devices (United States) and Lumen Photon Therapy (United States).

Many different examples of light therapy and PDT devices are known in the patent art. Early examples include U.S. Pat. No. 4,316,467 (Muckerheide) and U.S. Pat. No. 4,672,969 (Dew). The most common device design, which comprises a hand held probe, comprising at least one light emitter, but typically dozens or even 100 emitters, that is attached to a separate drive controller, is described in numerous patents, including U.S. Pat. No. 4,930,504 (Diamantapolous et al.); U.S. Pat. No. 5,259,380 (Mendes et al.); U.S. Pat. No. 5,464,436 (Smith); U.S. Pat. No. 5,634,711 (Kennedy et al.); U.S. Pat. No. 5,660,461 (Ignatius et al.); U.S. Pat. No. 5,766,233 (Thiberg); and U.S. Pat. No. 6,238,424 (Thiberg).

The light therapy devices that are commercially available today are disadvantaged in that the clinician does not know either the optical dosage delivered (light into the tissue) or the effective dosage delivered (light-tissue interaction). In part, the uncertainty is because many participants are not well educated in optics, and do not know how to measure light properly. However, the uncertainty is also because the science of light therapy is complicated. The leading theory for light therapy describes a process in which cytochrome oxidase (and other bio-chemicals), absorb incident light energy thus generating free electrons, which are then transferred within the mitochondrial electron transport chain to produce biochemicals such as adenosine triphosphate (ATP). ATP is then used in various cellular processes (including the synthesis of proteins and RNA). Additionally, various cell types (fibroblasts, epithelial cells, macrophages, mast cells, etc.) can apparently be stimulated for various effects, with these effects possibly occurring over hours, days, or even weeks. With these uncertainties, the clinician does not really know the efficacy of prior light applications relative to the response of the injured tissue, or whether further light application is appropriate, with what parameters, and for what effects.

Although very few light therapy devices actually attempt to provide a diagnostic feedback component, the problem has not gone unrecognized, and there are several prior art patents that propose solutions. Exemplary patents include U.S. Pat. No. 4,576,173 (Parker et al.) which includes monitoring of reactive oxygen species (ROS) in a photodynamic therapy (PDT) device, U.S. Pat. No. 4,930,504 (Diamantopolous et al.) which includes monitoring for skin temperature and trigger points, U.S. Pat. No. 4,973,848 (Kolobanov et al.) which includes using an analysis laser to induce tissue fluorescence for monitoring. As other examples, U.S. Pat. No. 5,755,752 (Segal) provides a light therapy dive using an impedance sensor to measure the DC resistance of the skin as a guide to treatment, U.S. Pat. No. 6,413,267 (Dumoulin-White et al.) provides a device equipped with detectors to measure scattered light as an indication of the depth of light penetration, and U.S. Pat. No. 6,663,659 (McDaniel) provides a light therapy device that employs an intra-dermal skin temperature probe. U.S. Pat. No. 6,676,655 (McDaniel) provides a light therapy device particularly targeted towards fibroblasts, which employs pulsed femtosecond yellow laser light (590 nm) to induce stimulatory effects. However, none or these prior art light therapy devices utilize diagnostic methods or devices that examine the structure and integrity of the extracellular matrix, in particular relative the collagen network, whether with polarized light, or by other means.

### SUMMARY OF THE INVENTION

Briefly, according to one aspect of the present invention a polarization based diagnostic device for optically examining the medical condition of tissue comprises an illumination optical system, comprising a light source having one or more light emitters, and beam shaping optics, which together provide illumination light to the tissue being examined. An optical detection system comprises imaging optics and an optical detector array detects light from the tissue. Polarizing optics provided in both the illumination optical system and the optical detection system are crossed so as to pass orthogonal polarization states. Iterative rotational means are provided to rotate the orthogonal polarization states relative to the tissue being examined. Image enhancement means includes some combination of image processing, sequential multi-spectral illumination and imaging, and image focus control to facilitate quality imaging at varying depths within the tissue. A controller operates the light source, the detector array, the multi-spectral illumination and imaging, and the image focus control, as well as providing image processing of the captured images to aid the diagnostic process.

An object of the present invention is to optically examine the medical condition of tissue.

These objects are given only by way of illustrative example, and such objects may be exemplary of one or more embodiments of the invention. Other desirable objectives and advantages inherently achieved by the disclosed invention may occur or become apparent to those skilled in the art. The invention is defined by the appended claims.

### BRIEF DESCRIPTION OF THE DRAWINGS

The foregoing and other objects, features, and advantages of the invention will be apparent from the following more particular description of the embodiments of the invention, as illustrated in the accompanying drawings. The elements of the drawings are not necessarily to scale relative to each other.

FIG. 1 is a cross-sectional view of the epidermal and dermal layers of the skin.

FIG. 2 is a histological cross-sectional picture of a tissue sample, showing a fibroblast and collagen structures.

FIGS. 3a and 3b are two histological cross-sectional picture showing collagen structures in skin.

FIG. 4 is an illustration of Langer's cleavage lines.

FIG. 5 is a picture of a pressure ulcer.

FIG. 6 is a side view of the general concept for the polarization diagnostic device of the present invention

FIGS. 7a, 7b, and 7c are side views of different conceptual embodiments for the polarization diagnostic device of the present invention.

FIG. 8 depicts a wire grid polarizer, which can be used in this invention.

FIG. 9 depicts a light therapy system used in conjunction with the polarization diagnostic device of the present invention.

### DETAILED DESCRIPTION OF THE INVENTION

The following is a detailed description of the preferred embodiments of the invention, reference being made to the drawings in which the same reference numerals identify the same elements of structure in each of the several figures.

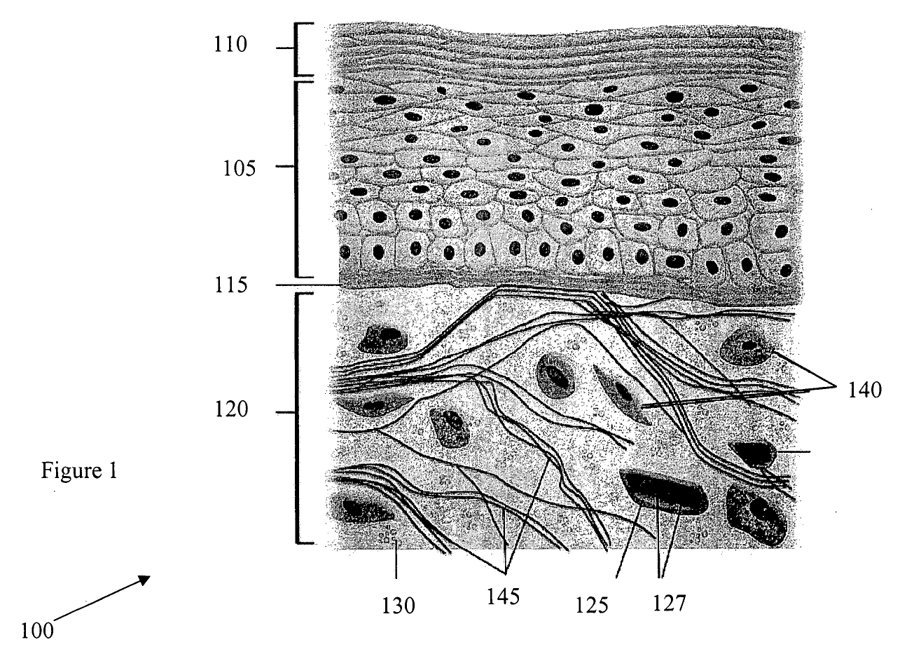

The present invention can be best appreciated within the context of the biology of normal, wounded, and healed skin, and in particular, with respect to the function of fibroblasts and collagen. Accordingly, FIG. 1 depicts the cross-sectional composition of skin. Skin 100 (or the integument) covers the entire external surface of the human body and consists of two mutually dependent layers, the epidermis 105 and the dermis, which rest on a fatty subcutaneous layer, the panniculus adiposus (not shown). The epidermis 105, which is the outer layer of skin, is made up of epithelial cells (also known as squamous cells or keratinocytes), basal cells, and melanocytes. The outermost layer of the epidermis 105 comprises layers of dead epithelial cells 110. The basal cells are responsible for producing the epithelial cells, while the melanocytes produce pigments (melanin) that give skin its color. Below the epidermis 105 is the basement membrane 115 (also known as the basal lamina), which helps attach the epidermis 105 to the reticular dermis 120. The basal lamina 115 actually comprises several layers, and includes proteoglycans and glycoproteins as well as Type IV collagen. The innermost layer of the basal lamina 115 includes several types of fibrils, including collagen type III and type VII fibrils, which help anchor to the dermis. The dermis comprises several layers, including the papillary dermis (not shown) and the reticular dermis 120, which is the primary dermal layer. The papillary dermis is composed of fine networks of types I and III collagen, elastic fibers, ground substance, capillaries and fibroblasts. The reticular dermis 120 contains thick collagen bundles (thicker than the papillary dermis), which are arranged in layers parallel to the surface of the skin. In FIG. 1, the reticular dermis 120 is shown, with constituent blood capillaries 125 with transiting red blood cells 127, fibroblasts 140, collagen fiber bundles 145, and proteoglycans 130. Proteoglycans 130 are large molecules that attract and hold water, thereby providing cushioning and support. The reticular dermis 120 also contains other structures (not shown), such as elastin, sebaceous glands, sweat glands, hair follicles, and a small number of nerve and muscle cells.

The dermal skin layers vary with body location. For example, skin is quite thin on the eyelids, but is much thicker on the back and the soles of the feet. The epidermis ranges in thickness from ˜30 microns to ˜1 mm, while the dermis (papillary and reticular) ranges between ˜300 microns and ˜3 mm in thickness. The collagen structure in skin also varies with location, as will be discussed subsequently.

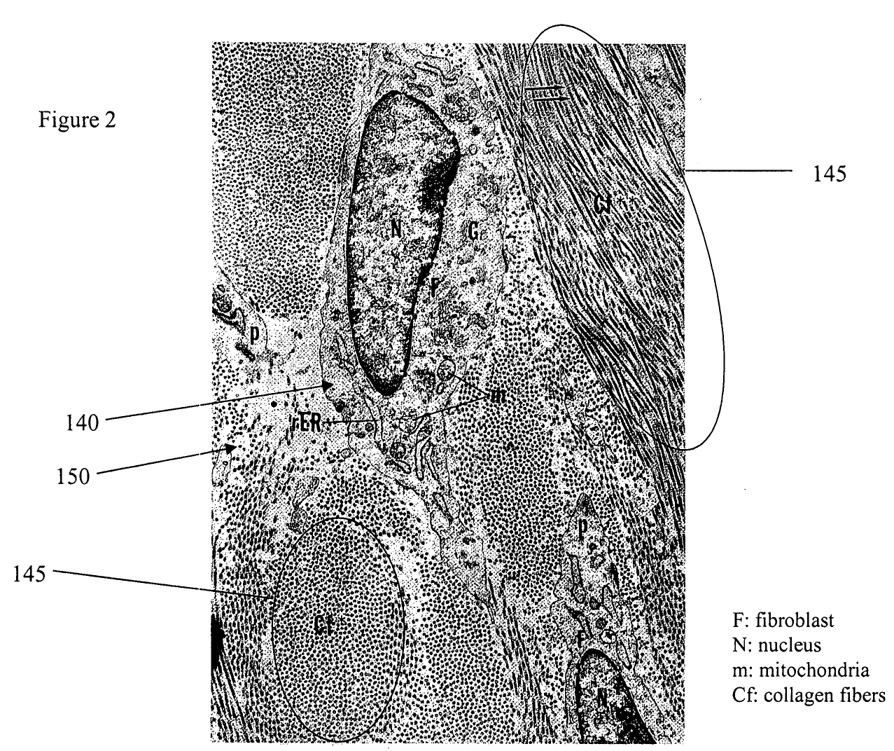

Fibroblasts create many of the components of the connective tissue in the reticular dermis, including the elastin, fibronectin, and collagen, which are all complex fibrous proteins. Collagen actually comprises long bundles or strands, composed of innumerable individual collagen fibrils. A fibroblast 140, is depicted in a histology image in FIG. 2, with at least four collagen fiber bundles 145, comprising numerous individual collagen fibrils 150, seen both in cross section and in plane within the image. Fibroblasts synthesize collagen (both Type I and Type III), in a process beginning with procollagen, which is polymerized outside the fibroblasts to form tropocollagen, which in turn is formed into collagen fibrils and collagen bundles. The collagen fibril segments are ˜25-50 microns in length and ˜10-200 nm in diameter (depending on type). These fibril segments fuse linearly and laterally (crosslink) to form longer, thicker, biomechanically competent collagen fibrils 150 within collagen bundles 145, which can be 200 microns in length. Smaller collagen bundles can be 0.5-10 microns in diameter, although thicker bundles, particularly in the reticular dermis, can be ˜100 microns in diameter. Notably, Type III collagen fibers are generally thinner than the Type I fibers.

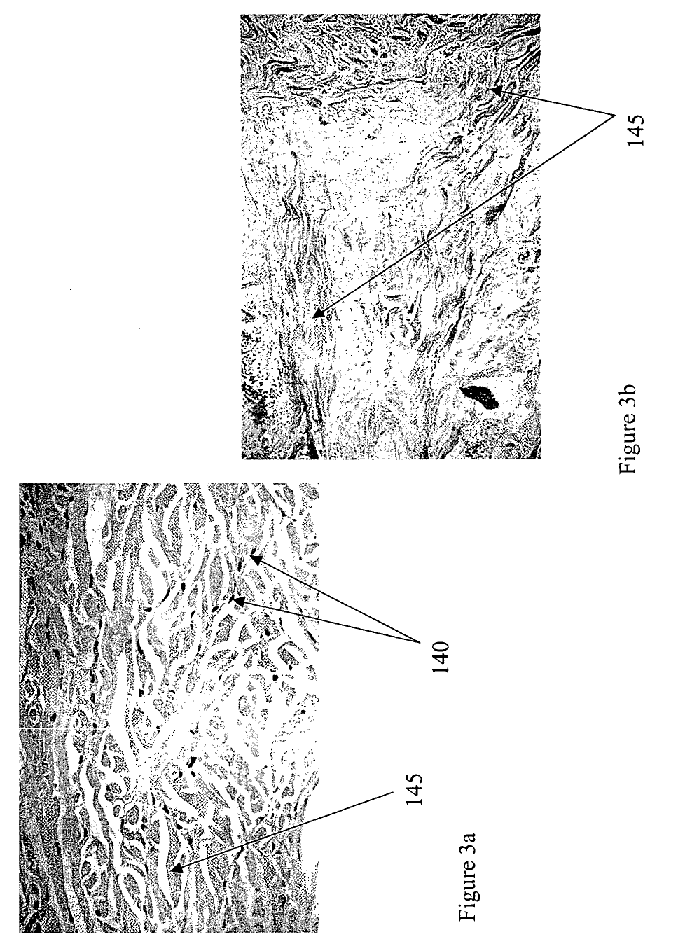

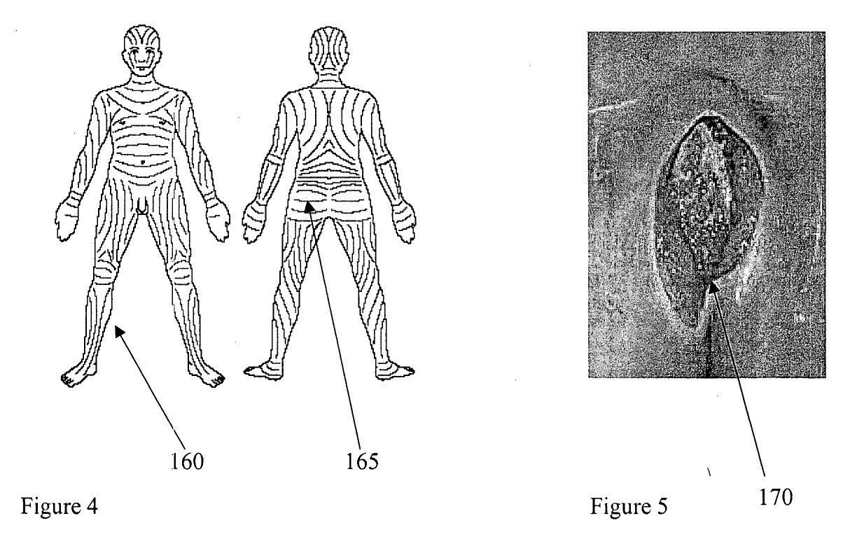

The most structured collagen formations are found in bones and tendons. The collagen structures in tendons, ligaments, and vocal cords, which are termed “dense regular” and have collagen fibers in parallel alignment, are structured to handle stresses and transmit forces along their length. By comparison, the collagen structures in skin (see FIGS. 3a and 3b), in which the collagen fibrils and bundles are less organized and somewhat wavy or convoluted, are termed “dense irregular.” Although some researchers have described the collagen structures in skin to be random or haphazard (see FIGS. 3a and 3b), there is both local and macro patterning. Human dermal tissue (skin) is compliant and adapts to pressures from all directions. The collagen network, which is multi-directional and multi-layered, is an interwoven mesh generally parallel to the surface of the skin, which gives skin its toughness and adaptability. However, there is a pre-dominant direction to the orientation of the fiber bundles in a given location. As shown in FIG. 4, Langer's cleavage lines 165 are generally associated with the alignment of collagen bundles deep in the reticular dermis. These lines portray the directional effects of skin across the human body 160, wherein the stress-strain relationships in uniaxial tension show skin to be stiffer along Langer's lines than across the lines. Langer's lines 165 are used as guides in surgery, with incisions preferentially running along the lines rather than cutting obliquely through them. This is because incisions along these lines heal with a minimum of scarring, whereas oblique wounds may be pulled apart or develop thicker scars. Collagen bundles that follow Langer's lines may be several millimeters, or even a centimeter or more in extent. Some common directionality, at least on a local scale of a few hundred microns, is evident in the collagen structures in the skin of FIGS. 3a and 3b. Collagen fibers generally do not often branch and, when branches are found, they usually diverge at an acute angle (see FIG. 1).

The natural mesh-like arrangement of collagen fibers in skin allows continual rearrangement of individual fibers to resist severe stretching under the minimal stresses associated with normal activity. At rest, the collagen fibers are irregularly organized, but when an increasing load is applied, the fibers change geometrical configuration and become parallel. The interconnected elastin fibers are able to stretch much more than the collagen fibers, and likely assist the collagen fibers to return to their original alignment after the forces have been removed. The water, proteins, and macromolecules (proteoglycans) function as a lubricant during deformation.

Wounds are characterized in several ways; acute wounds are those that heal normally within a few weeks, while chronic wounds are those that linger for months or even years. Wounds that heal by primary union (or primary intention) are wounds that involve a clean incision with no loss of substance. The line of closure fills with clotted blood, and the wound heals within a few weeks. Wounds that heal by secondary union (or secondary intention) involve large tissue defects, with more inflammation and granulation. Granulation tissue is needed to close the defect, and is gradually transformed into stable scar tissue. Such wounds are large open wounds as can occur from trauma, burns, and pressure ulcers. While such a wound may require a prolonged healing time, it is not necessarily chronic. A chronic wound is a wound in which normal healing in not occurring, with progress stalled in one or more of the phases of healing. A variety of factors, including age, poor health and nutrition, diabetes, incontinence, immune deficiency problems, poor circulation, and infection can all cause a wound to become chronic. Typical chronic wounds are pressure, friction ulcers, and venous stasis ulcers. Chronic wounds are also categorized relative to the extent of the damage:
Stage 1—has observable alteration of intact skin with changes in one or more of skin temperature, tissue consistency, or sensation (pain, itching). Pro-active treatment of Stage 1 and Pre-Stage 1 (also known as Stage 0) wounds could be beneficial.
Stage 2—involves partial thickness skin loss involving epidermis, dermis, or both. The ulcer is superficial and appears as an abrasion, blister, or shallow crater.
Stage 3—Full thickness skin loss with damage or necrosis of subcutaneous tissue.
Stage 4—Full thickness skin loss with extensive destruction, tissue necrosis, and damage to muscle, bone, or supporting structures (tendon, joint, capsule, etc.). Successful healing of Stage 4 wounds still involve loss of function (muscles and tendons are not restored).
Stage 5—Surgical removal of necrotic tissue usually required, and sometimes amputation. Death usually occurs from sepsis.

Wound healing also progresses through a series of overlapping phases, starting with coagulation (haemostasis), inflammation, proliferation (which includes collagen synthesis, angiogenesis, epithelialization, granulation, and contraction), and remodeling. Haemostasis, or coagulation, is the process by which blood flow is stopped after the initial wounding, and results in a clot, comprising fibrin, fibronectin, and other components, which then act as a provisional matrix for the cellular migration involved in the later healing phases. Many of the processes of proliferation, such as epithelialization and angiogenesis (creation of new blood vessels) require the presence of the extracellular matrix (ECM) in order to be successful. Fibroblasts appear in the wound during that late inflammatory phase (˜3 days post injury), when macrophages release cytokines and growth factors that recruit fibroblasts, keratinocytes and endothelial cells to repair the damaged tissues. The fibroblasts then begin to replace the provisional fibrin/fibronectin matrix with the new ECM. The ECM is largely constructed during the proliferative phase (˜day 3 to ˜2 weeks post injury) by the fibroblasts, which are cells that synthesize fibronectin and collagen. As granulation continues, other cell types, such as epithelial cells, mast cells, endothelial cells (involved in capillaries) migrate into the ECM as part of the healing process.

Fibroblasts initial role in wound healing is to provide fibronectin, which is a glycoprotein that promotes cellular adhesion and migration. Fibronectin weaves itself into thread-like fibrils, with “sticky” attachment sites for cell surfaces, to help connect the cells to one another. There is some directionality to the deposition of fibronectin, which in turn impacts the deposition of the other ECM proteins. Fibroblasts synthesize collagen (both Type I and Type III), beginning with procollagen, which is three polypeptide chains (each chain is over 1400 amino acids long) wound together in a tight triple helix. Procollagen is then extruded from the fibroblast out into the extracellular space. Once exocytosed, these filaments lay disorganized in the wound, still in a gelatinous state. The triple-helical molecule undergoes cleavage at specific terminal sites. The helix is now called a tropocollagen molecule, and tropocollagens spontaneously associate in an overlapping array. The amassing continues as tropocollagen convolves with other tropocollagen molecules to form a collagen fibril. Wound durability, or tensile strength, is dependent on the microscopic welding (cross-linking) that must occur within each filament and from one filament to another. The collagen fibril segments are ˜25-50 microns in length and ˜10-200 nm in diameter (depending on type). The fibril segments fuse linearly and laterally (crosslink) to form longer, thicker, biomechanically competent collagen fibrils 150 within collagen bundles 145. Collagen deposition will align itself to the fibronectin pattern, which in turn mirrors the axis of the fibroblasts. Although the initial collagen deposition may appear somewhat haphazard, the individual collagen fibrils are subsequently reorganized, by cross-linking, into more regularly aligned bundles oriented along the lines of stress in the healing wound, and eventually, at least partially, to the stress lines associated with the surrounding tissue.

Type III collagen is the type that appears in the wound initially, starting at about 4 days after injury. Collagen becomes the foundation of the wound ECM, and if collagen formation does not occur, the wound will not heal. Myofibroblasts, which are a specialized fibroblast, appear late during the proliferative phase (at ˜5 days), to help contract the wound so that there will be less scarring. Wound contraction helps to further organize the early collagen structures. A ring of these contractile fibroblasts convene near the wound perimeter, forming a “picture frame” that will move inward, decreasing the size of the wound. Linear wounds contract rapidly, square or rectangular wounds contract at a moderate pace, and circular wounds contract slowly.

As wound healing progresses into the remodeling stage (starting at ˜10 days post injury) the fibroblasts continue to work to build more robust tissue structures. Matrix synthesis and the remodeling phase are initiated concurrently with the development of granulation tissue and continue over prolonged periods of time (˜30-300 days, depending on the injury). As the extracellular matrix matures, fibronectin and hyaluronan (a component of the proteoglycans) are broken down. Over time, fibronectin is replaced by Type III collagen and ultimately by Type I collagen. Type III collagen is fairly quickly replaced by Type I collagen, which constitutes 90% of the total collagen in the body, and forms the major collagen type found in the reticular dermis. As remodeling progresses, towards a goal of having the new ECM match the original and fit with the surrounding tissue, the collagen structure is altered on an ongoing basis, by a process of lysis and synthesis. Collagen degradation is achieved by specific matrix metalloproteinases (MMPs) that are produced by many cells at the wound site, including fibroblasts, granulocytes and macrophages. Gradually, the Type I collagen bundles are deposited with increasing organization, orientation, and size (including diameter), to better align to the surrounding tissues and increase wound tensile strength.

An ideal case of wound healing is one in which there is a complete regeneration of lost or damaged tissue and there is no scar left behind. In the case of a minor acute wound, which heals by primary intention, there will be little or no scarring, and the final tissue will be basically equivalent to the original. In the cases of an acute wound that heals by secondary intention (multiple layers of skin are injured), the healed wound will likely include some portion of scar tissue. Scars start as granulation tissue with large irregular mass of collagen. As with the primary union degree wound, scar remodeling for a secondary union type wound continues, attempting to mimic the surrounding tissue in structure and strength. The amount of scar to be remodeled is inversely related to the return of function. However, typically the fully healed scar has only 70-80% of the strength of the original tissue. In part this is because the collagen bundles never match fully match the original, nor regain the original alignments. Additionally, as adults produce few new elastin fibers during healing, the scar lacks the elasticity and recoil of the original tissue.

As previously stated there are several types of chronic wounds, including the pressure ulcer (or decubitis ulcers or bed sores), all of which suffer impaired healing. Stage 3 and Stage 4 pressure ulcers (see FIG. 5) are open wounds that can occur whenever prolonged pressure is applied to skin covering bony outcrops of the body. Patients who are bedridden are at risk of developing pressure ulcers. Stage 4 pressure ulcers can form in 8 hours or less, but take months or years to heal. Pressure ulcers 170 are complicated wounds, which can include infection, slough (dead loose yellow tissue), black eschar (dead blackened tissue with a hard crust), hyperkeratosis (a region of hard grayish tissue surrounding the wound), and undermining or tunneling (an area of tissue destruction extending under intact skin). Pressure ulcers may have closed wound edges (epibole), which impedes healing. In such circumstances, the top layers of the epidermis have rolled down to cover lower edge of epidermis, including the basement membrane, so that epithelial cells cannot migrate from wound edges. The efforts of the fibroblasts and the myofibroblasts to build the ECM and close the wound can be exhibited in a “collagen ridge” or “healing ridge,” which is a region surrounding the wound (extending perhaps ˜1 cm on each side) where new collagen synthesis is occurring. During treatment, clinicians often have to locate the collagen ridge by feel (palpitation), in order to assess the wound condition and treatment. However, the collagen ridge may be poorly defined and difficult to locate. The collagen in healing pressure ulcer tissue is different than that in normal tissue, as there are fewer collagen fibers, but they may be significantly wider and longer than in normal tissue.

As can now be appreciated, successful collagen formation and remodeling is very important in wound healing, whether the wounds are acute (primary or secondary) or chronic, and whether the wounds are in the inflammatory phase, the proliferative phase, or the remodeling phase, or a combination thereof. In the case of chronic wounds, it could be valuable to have a device to detect the collagen ridge structure in a Stage 3 or Stage 4 wound. It could also be valuable to have a collagen detection device that would facilitate detection of Stage 1 and Pre-Stage 1 wounds, by revealing collagen structure degeneration. In that case, pre-emptive treatments could be attempted before the skin ruptures, which could greatly improve outcomes. Typically today, clinicians are not reimbursed for treatment of Stage 1 and Pre-Stage 1 conditions, as there are only subjective or visual measures available for tissue condition, rather than any quantitative measures. The polarization diagnostic device of the present invention does not need to he limited to examining the collagen network, as a means for determining tissues status. Both elastin and fibronectin, which are elongated thread like proteins, are likely optically birefringent and could potentially be detected. As fibronectin is deposited prior to collagen Type I, detection of fibronectin would enable examination at an earlier point in the healing process. It is also noted that there are actually 14 different types of collagen. While collagens Types I and III are pre-dominant in the skin, the other collagens, which may also be optically birefringent, can be found in other biological structures. As an example, capillaries, which are tubules that are constructed in part with Type IV collagen, are said to be optically birefringent. Detection and tracking of capillary formation (angiogenesis) with the device of the present invention in tissue undergoing granulation and remodeling could also be useful in understanding tissue status. Additionally, muscles (which comprise a birefringent filamentous protein f-actin), nerves (which includes sheaths of birefringent myelin covering the axons), and amyloids (starch like birefringent proteins that aggregate and impair function, for example in Alzheimer's disease) might all be examined using the device of the present invention.

The birefringent structures (collagen included) in the tissue can potentially be monitored with polarization optics that enable examination of the optically birefringent structure of the collagen. Isotropic (homogeneous) media (such as glass) have a single index of refraction. Anisotropic media may have either two or three indices of refraction. Uniaxial media (such as liquid crystals) have two indices of refraction, which are the ordinary index (no) the extraordinary index nₑ. The axis of nₑ is also referred to as an optical axis. Uniaxial materials are uniquely characterized by nₑ, nₒ, and two angles describing the orientation of its optical axis. Optical materials with all three different refractive indices are called biaxial, and are uniquely specified by its principal indices nx₀, ny₀, nz₀, and three orientational angles.

Light sees varying effective indices of refraction depending on the polarization direction of its electric field when traveling through an anisotropic material, and consequentially, a phase difference is introduced between two eigen-modes of the electric field. This phase difference varies with the propagation direction of light, so the transmission of the light varies with angle when uniaxial or biaxial materials are placed between two crossed polarizers. It is generally understood that retardance is the delay of one polarization relative to the orthogonal polarization, where the delay translates into a phase change Δφ in the polarization of the incoming light. The phase change Δφ can be calculated as
Δφ=2π*t*Δn/λ,
where (Δn) is the index change (Δn=n_{∥}−n_{⊥}=nₑ−nₒ) (intrinsic birefringence) provided by the structure and (t) is the thickness of the structure. Retardance is the phase change Δφ expressed as distance; for example a π/2 phase change Δφ corresponds to a quarter wave λ/4 retardance, which at 550 nm equals ˜138 nm retardance. These phase differences translate into modifications of the local polarization orientations for rays traveling along paths other than along or parallel to the optical axis. When viewed under polarized light, however, anisotropic materials will be brightly visible in one plane (“birefringent”), but will be dark in a plane turned 90 degrees. The refractive index of human tissue (collagen included) is n ˜1.4-1.5, depending on the tissue and the wavelength. Both Type I and Type III collagens are birefringent, with nominal optical birefringence values of Δn˜3×10⁻³.

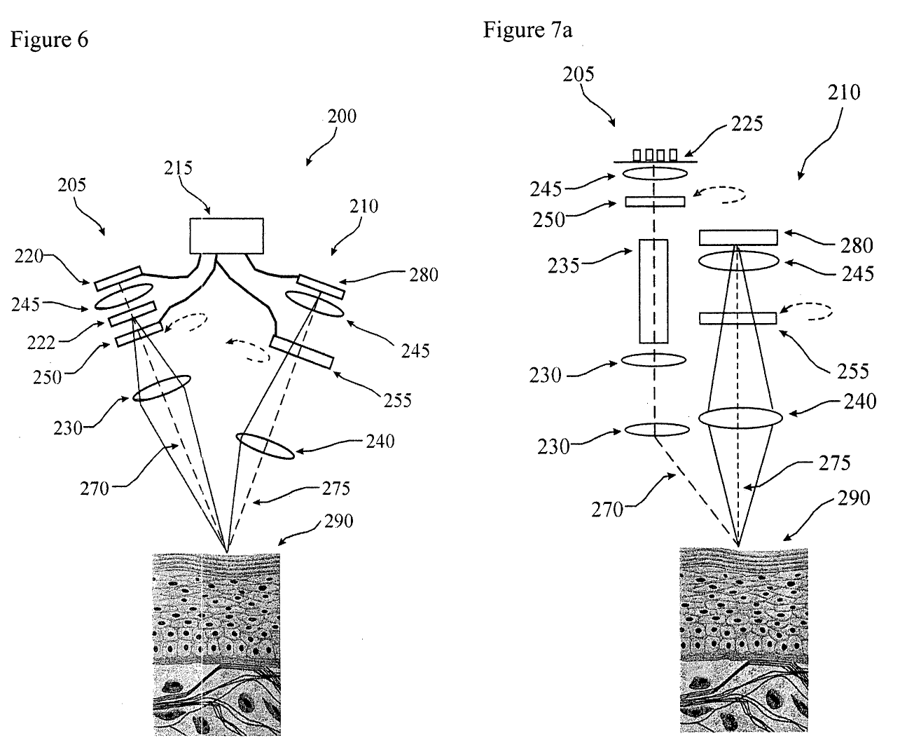

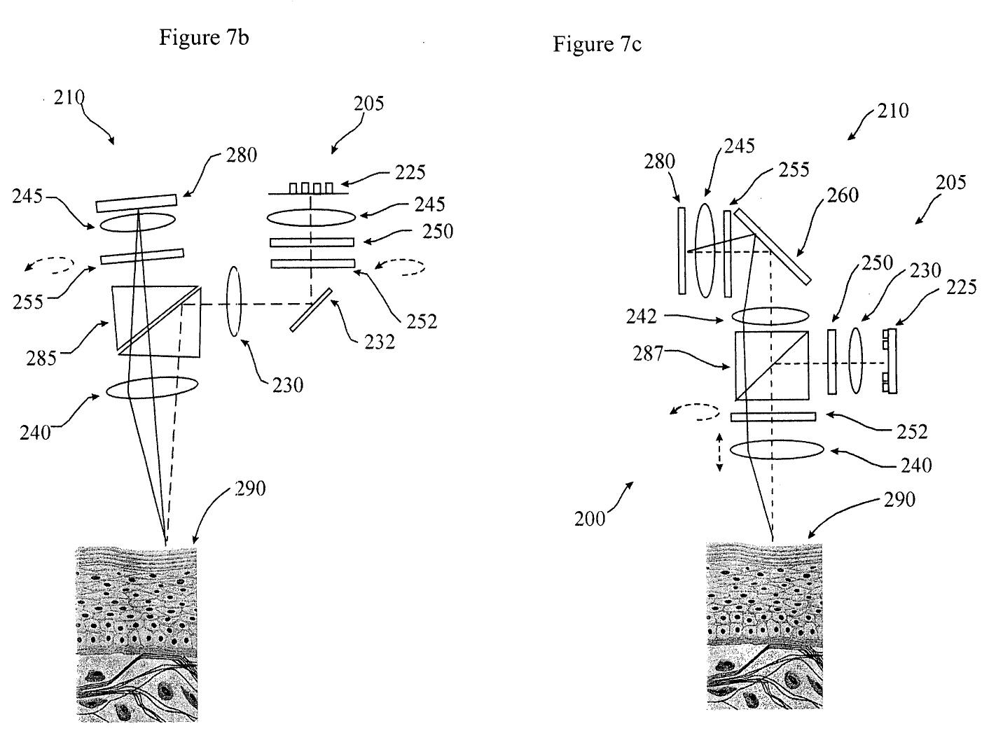

The present invention then provides a diagnostic device for examining the state of dermal tissue, that is optimized for examining birefringent structures in tissue, and in particular for examining collagen structures in the extra-cellular matrix of the dermis. The basic device concept is shown in FIG. 6. A polarization diagnostic device 200 comprises an illumination system 205 and a detection system 210 (linked by a controller 215), which are both directed at the same nominal portion of tissue 290. Note that the FIG. 6 (and FIGS. 7a, 7b, and 7c) are not to scale; the optical systems likely measure several inches end to end, but the depth of the tissue examined is only ˜2-4 mm. In the conceptual device of FIG. 6, both the illumination system 205 and the detection system 210 are aimed obliquely at the tissue 290. The illumination system 205 nominally comprises a light source 220 and illumination beam shaping optics. The beam-shaping optics can comprise a condenser lens 230, a pre-polarizer 250, spectral filters 222, light uniformization optics (such as a Fly's eye integrator or integrating bar, but not shown), and field lenses (such as field lens 245), as well as other components. In this system, condenser lens 230 can image the front focal plane of the field lens 245, so that a Koehler type illumination system is provided as a means of providing reasonable illumination uniformity to the tissue 290. In general it is assumed that illumination system 205 is illuminating an area on the tissue which is at minimum ˜10 mm², but could be ˜1.0 in² or more. Optimally, illumination system 205 illuminates an area of tissue larger than what is imaged to detector 280. Light source 220 can be a lamp (such as tungsten halogen, metal halide, or UHP), an LED (light emitting diode), a SLD (super-luminescent diode), a laser diode, or other light source.

Optical detection system 210 nominally comprises an objective lens 240 that provides an image of the tissue 290 on detector 280. Detector 280 is nominally a detector array, such as a charge coupled device (CCD) or a complementary metal oxide semiconductor (CMOS) device. Detector 280 is nominally an area device with a row and column structure. An exemplary device could be the Kodak KAF-6303, which comprises an array of 3072×2048 pixels, with a nominal 9 micron pixel pitch. Incident light provided by the illumination system 205 will penetrate the tissue 290. Some portion of this incident light will be reflected or backscattered from the various tissue components (organelles, cells, and extra-cellular components such as collagen) it encounters and can be imaged by objective lens 240 onto detector 280. For example, if objective lens 240 imaged the tissue onto the 6 Mpixel KAF-6303 detector with 1× magnification, an area of the tissue ˜18.4 mm×27.6 mm could be examined at one time. Polarization diagnostic device 200 is nominally equipped with at least two linear optical polarizers, pre-polarizer 250 and polarization analyzer 255 that are provided to enable detection of the birefringent tissue structures. Pre-polarizer 250 rotates around the illumination optical axis 270, while polarization analyzer 255 rotates about the imaging optical axis 275. These two polarizers are nominally orthogonal to their respective axes, although they may be tilted (likely by a few degrees) away from orthogonality, to control the direction of any ghost reflections, to thereby improve image contrast. That is, pre-polarizer 250 and polarization analyzer 255 are nominally crossed (90 degrees rotationally apart) to define extinction axes. Light from the illumination system 205 is then incident on the tissue 290 with an initial linear polarization alignment. Some of this light will penetrate the tissue 290, and another portion will be specularly reflected from the first surface of the tissue. This specularly reflected light tends to retain the polarization state of the illumination light. Light that penetrated tissue 290 and then re-emerges while nominally retaining the initial polarization state will be eliminated by crossed polarization analyzer 255 and not reach detector 280, and therefore not provide an effective image. Likewise, the polarization analyzer 255 will also eliminate the specularly reflected light from the first surface of the tissue. Whereas, light that re-emerges from tissue 290 with its polarization rotated to some extent by the birefringent structures within the tissue, can then have some portion of that light transmitted through polarization analyzer 255 and thus imaged at detector 280. Re-emergent light that has a polarization vector orthogonal to the illumination polarization axis, and therefore nominally aligned to the polarization axis of the analyzer 255 will be imaged with maximal image brightness.

In this way, the polarization sensitive optics enable the imaging of the birefringent tissue structures by enabling detection of changes in the polarization state of the low level diffused light re-emerging from the tissue, while eliminating the strong initial back reflection off of the front surface, which could otherwise provide a dominant return signal and reduce the contrast of the images of the birefringent tissue structures.

Recall that the collagen network in relaxed skin likely has local directional variations (see FIGS. 3a and 3b), birefringence is spatially variant. Therefore, the image quality of the collagen network depends on the relative alignment of the crossed polarizers (250 and 255) to any given portion of the network. Thus to improve the quality of the images of the collagen network, the present invention anticipates that the crossed polarizers should be rotated in unison so that the extinction axes rotate into various positions relative to the tissue 290. This is facilitated by controller 215, which sends drive signals to mechanisms (not shown), such as stepper motors, which separately drive pre-polarizer 250 and polarization analyzer 255 to rotate about their respective optical axes. Nominally crossed polarizers 250 and 255 each rotate by the same angular amount Δφ, so that they remain crossed. Crossed polarizers 250 and 255 nominally are rotated in a stepwise fashion through N steps, of some set amount Δφ, until the crossed polarizers have both swept through at least 90 degrees. For example, crossed polarizers could start at some set of initial positions, and then be synchronously swept through N=6 steps, so that Δφ=15°, and the polarizer step through relative angular positions of 0°, 15°, 30°, 45°, 60°, 75°, and 90°. At each step, controller 215 would drive light source 220 to provide illumination light and detector 280 to capture a digital image. Controller 215 could, for example store each of these images, and then directly present them to the clinician (for example by a built in LCD panel) for evaluation. Alternately, controller 215 could employ image-processing algorithms to build one or more composite high contrast images. These image-processing algorithms could perform various functions (sharpening, contrast changes, false color, etc.) to enhance image quality/wound visualization. The algorithms could also calculate and display some quantitative metrics for each image, as well as the ensemble thereof, that indicate the relative conformity of the collagen network (for example the number of areas (of some size or % image field) having a common directionality (for example, relative to some statistical measure). The timing of the iterative, step-wise, rotation of crossed polarizers 250 and 255 will likely be determined by the image capture and processing times needed by the controller 215 to assemble and analyze the acquired images.

As previously stated, there are prior art optical devices that enable examination of birefringent structures in tissues. For example, the previously discussed polarization OCT systems offer good optical resolution (˜5-10 microns), good polarization sensitivity and dynamic range (˜50-120 dB), and the ability to control image focus to various depths (by interferometry) within the tissue. Unfortunately, OCT systems are too large and too costly to be used in many clinical settings, including in the field. However, the in-vivo polarization diagnostic device 200 of the present invention would be more valuable if it matched or approached some of this functionality of the OCT systems. The present invention can include several design aspects to improve both the potential performance and operation of a device following the general concept described in FIG. 6, including application of image processing software, the use of multi-spectral imaging, focus control, and high contrast (dynamic range) optics, and the design of a more compact device. As will be seen, the particular combination of multi-spectral imaging and polarization imaging anticipated by the present invention may be especially advantageous for examining spatially complex birefringent tissue structures.

To begin with, it was previously noted that the collagen network is present at various depths within the reticular dermis. It may be valuable to examine the formation of the collagen network (and other birefringent structures) at different depths in the tissue. Unfortunately, the window of opportunity for optical imaging is not particularly wide. It is well known in photobiology, that there is an optical transmission spectral window in which light achieves its deepest penetration, which spans the red and near infrared spectrum from ˜620 nm to ˜1300 nm. In particular, light between ˜650 nm and ˜1000 nm can penetrate the deepest into human tissue (˜20% incident light can reach ˜3.5 mm depth). An approach that utilizes multi-spectral imaging to see tissue structures at different depths could be particularly valuable. For example, light source 220 could sequentially provide illumination light with an increasing nominal wavelength, starting at ˜530 nm (to image structures within the first ˜0.5 mm tissue depth), then ˜600 nm (to image structures within the first ˜1 mm tissue depth), then ˜630 nm (to image within the first ˜2 mm tissue depth), and ˜830 nm light (to image within the first ˜3.5 mm tissue depth). Then for each rotational position of the crossed polarizers 250 and 255, controller 215 could capture digital images for each tissue depth. Controller 215 could then present the clinician each image to view by a display. However, the device 200 may be more valuable if the image-processing algorithms within controller 215 could calculate metrics for each image (such as amount and conformity and extent of birefringent structures) and apply shallower images to the original data from deeper images, to remove scatter and birefringence effects from the shallower images. In that way, the device provides a form of spectral polarization difference imaging, through which truer corrected digital images of the birefringent structure of the tissues at various levels may be extracted. In some cases, the birefringent tissue may be so uniformly aligned over tissue area and depth, that a single spectral image with minimal or no corrections from shallower images may well represent the status of the tissue.

If the light source 220 is a broadband source (such as tungsten halogen bulb lamp), spectral filtering will be required. For example, illumination system 205 of FIG. 6 could be provided with a fixed spectral filter 222 (to block light from outside the desired spectrum (˜550 nm to 1000 nm)), as well as narrow spectrum notch filters. For example, there could then also be a series of fixed spectral notch filters 222 (for each wavelength (530, 600, 630, and 830 nm), each with a narrow bandpass (˜15 nm, for example), that could be mounted on a filter wheel. However, as a filter wheel might be a large and cumbersome mechanism for this device, other solutions could be useful. For example, a liquid crystal tunable filter could be used. An exemplary device is the near IR version of the Varispec filter, from CRI Inc. (Woburn, Mass.) that can be controlled to provide narrow transmission spectra (such as 10-20 nm wide) within a spectral band covering 650-1100 nm. Controller 215 would also control the operation of the variable spectral filter. Obviously, to minimize the operational time for the device to collect and process images for a given tissue location, it would be preferable if the capture time was minimized, which in turn means that the number of sequential illumination wavelengths used should likewise be minimized.

Alternately light source 220 could comprise an array of discrete light emitters, such as LEDs and/or laser diodes, with one or more light emitters provided for each wavelength of interest. As an example, the device of FIG. 7a depicts a light source comprising multiple light emitters 225. For example, Lumileds (San Jose, Calif.) and Osram Opto-Semiconductors (Regensburg, Germany) offer a range of visible and infrared LED that could be used for this application. In this case, controller 215 could sequentially drive the visible and infrared LEDs in order to provide the multi-spectral illumination (nominally wavelength sequential), imaging, and image processing. This is likely an improvement over the use of a broadband light source, as the LEDs are compact and emit over limited bandwidths, thus a fixed spectral filter 222 is likely not needed. If the LED emission bandwidth is sufficiently narrow to obtain good polarization imaging at the target tissue depths, the system can also be constructed without a variable spectral filter 222 (such as the liquid crystal tunable filter mentioned previously). However, a spectral filter 222 with variable spectral control can certainly be used in conjunction with the LEDs, and can be positioned either in the illumination system 205 (as shown) or in the detection system 210 (not shown).

The choice of polarization optics used in the polarization diagnostic device of the present invention is also critical. Many prior art systems have been described which use MacNeille type thin film prisms (U.S. Pat. No. 2,403,731), pulled polymer sheet polarizers (“Polaroid” polarizers), or bulk birefringent crystalline prisms (such as calcite). In recent years, visible wavelength wire grid polarizers have been developed. These polarizers, which are available from Moxtek (Orem, Utah), and which are described in U.S. Pat. No. 6,122,103 (Perkins et al.) and U.S. Pat. No. 6,243,199 (Hansen et al.), have many admirable features, including a broad spectral response, a broad angular response, high contrast, and good transmission (˜90%). In the main, these inexpensive devices are being used for image projection systems with liquid crystal displays (LCDs), where it is important to obtain high polarization contrast as well as good transmission with a fast (˜F/2.4) optical system. Exemplary systems and wire grid devices have been described, for example in U.S. Pat. No. 6,532,111 (Kurtz et al.), U.S. Pat. No. 6,585,378 (Kurtz et al.), and U.S. Patent Application Publication No. 2003/0128320 (Mi et al.) (all assigned to the same assignee as the present invention) in which wire grid polarizers were applied in projection systems intended to provide projected contrast of >1000:1. Indeed, operational systems have been described in the literature, in which projected image contrast levels >4000:1 have been reported.

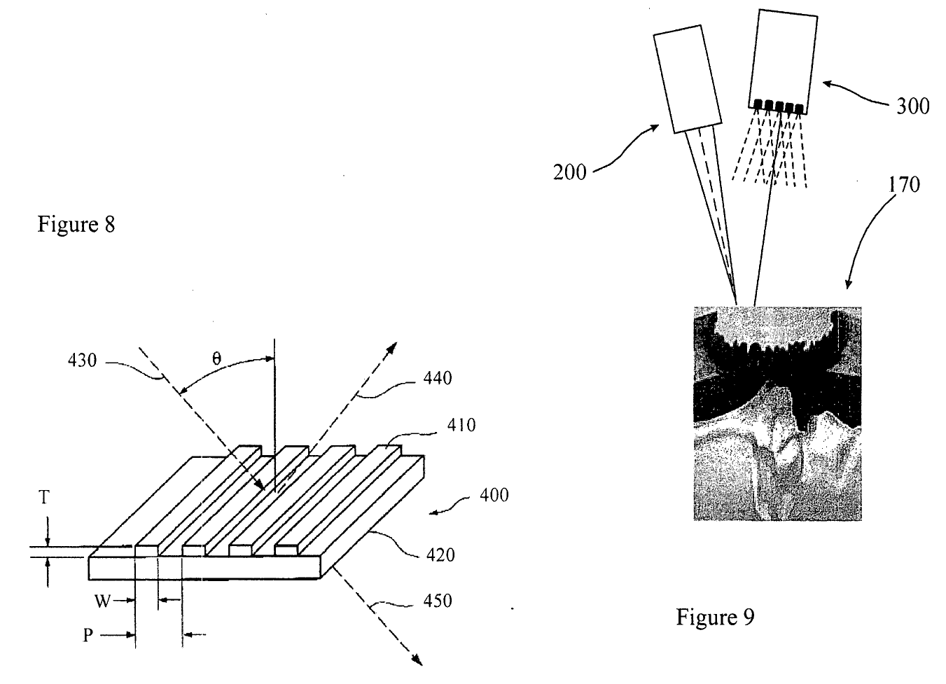

The wire grid polarizer can be better understood with reference to FIG. 8. The wire grid polarizer 400 is comprised of a multiplicity of parallel conductive electrodes (wires) 410 supported by a dielectric substrate 420. A beam of light 430 is nominally incident on the polarizer at an angle θ from normal, such that wire grid polarizer 400 divides this beam into specular non-diffracted outgoing light beams; reflected light beam 440 and transmitted light beam 450. When such a device is used at normal incidence (θ=0 degrees), the reflected light beam 440 is generally redirected towards the light source, and the device is referred to as a polarizer. However, when such a device is used at non-normal incidence (typically 30°<θ<60°), the illuminating beam of light 430, the reflected light beam 440, and the transmitted light beam 450 follow distinct separable paths, and the device is referred to as a polarization beamsplitter.

A wire grid polarizer device is characterized by the grating spacing or pitch or period of the conductors, designated (p); the width of the individual conductors, designated (w); and the thickness of the conductors, designated (t). Nominally, a wire grid polarizer uses sub-wavelength structures, such that the pitch (p), conductor or wire width (w), and the conductor or wire thickness (t) are all less than the wavelength of incident light (λ). The performance of wire grid polarizers, and indeed other polarization devices, is mostly characterized by the contrast ratio, or extinction ratio, as measured over the range of wavelengths and incidence angles of interest. For a wire grid polarizer or polarization beamsplitter, the contrast ratios (which depend on the p/λ ratio) for the transmitted beam (Tp/Ts) and the reflected beam (Rs/Rp) may both be of interest. The commercially available devices from Moxtek have a wire pitch p˜140 nm (λ/3), which makes these device sub-wavelength for blue light. As a result, this same device is −λ/6 for IR light, and thus the polarization contrasts (both transmitted and reflected) should be higher than in the visible (unless absorption by the metal wires increases).

Thus, it is realistic to utilize wire grid polarizers for the polarization diagnostic device 200 of the present invention. The 4000⁺:1 contrast reported in white light projection systems using a subsystem of wire grid polarizers equates to a dynamic range of 72 dB. Given that these same visible wavelength wire grid polarizers should have higher contrast for infrared operation, and further that the instantaneous bandwidth is less than in projection, the dynamic range could expand further. Certainly, polarization dynamic range values of 72 dB or greater are generally comparable to the dynamic range levels reported in OCT and polarization microscope systems (˜55-120 dB). This polarization dynamic range is only useful if the dynamic range of the detector 280 is at least comparable, if not larger. However, the exemplary KAF-6303 sensor, which limits the sensor noise sufficiently to provide a dynamic range of 76 dB, shows that this can be achieved, assuming that there is sufficient light to fill the electron wells during the sensor integration time. It should also be noted that the device of the present invention can be designed with a multi-color detector array, or with multiple detector arrays 280. For example, the device might be equipped with an IR optimized detector and a green/red optimized detector, so that each spectral region provides images with maximal dynamic range.

Relative to the polarization diagnostic device 200 of FIG. 6, it should be understood that both pre-polarizer 250 and polarization analyzer 255 are preferentially wire grid polarizers. Of course, several variations on the theme are possible. For example, polarization analyzer 255 might actually be two consecutive wire grid polarizers, with the second one provided to remove residual leakage light from the first, and thus enhance the contrast. In that case, both polarizers would rotate together as a pair, although the two polarizers might be tilted relative (by a few degrees, or near parallel) to each other to control ghost reflections. On the other hand, in the circumstance that light source 220 emits polarized light (for example if it comprises one or more laser diodes, then pre-polarizer 250 could be replaced with a waveplate (nominally λ/2 or λ/4) which would rotate the polarization state of the incident light relative to tissue 290. However, if the light source 220 emits unpolarized light, it could be disadvantageous to build the illumination system 205 with a pre-polarizer without first (or instead) providing a polarization conversion device, as otherwise as much as 50% of the available light will be lost right up front. While there are many polarization conversion designs known in the art, one particularly advantageous design and compact is described in U.S. Pat. No. 5,978,136 (Ogawa et al.), which uses an array of mini-prisms and waveplates to provide an ensemble of polarized light beams. Certainly other polarizer technologies with high contrast and transmittance, as well as a large angular acceptance, could be used in place of wire grid polarizers. For example, another candidate technology is the photonic crystal polarizer, which theoretically has an excellent field-of-view and wavelength acceptance. Such devices are available from Photonic Lattice Inc. (Japan). However, photonic crystal polarizers are presently fabricated using expensive lithographic processes.

One problem with the conceptual polarization diagnostic device of FIG. 6 is that it is not necessarily compact. FIG. 7a depicts an alternate device that could be more compact. The device of FIG. 7a (for simplicity shown without controller 215) provides illumination system 205 and detection system 210 with parallel optical paths, which could be assembled together in one housing (not shown). In this case, detection system 210 is shown to be nominally orthogonal to the tissue 290, which is a preferable circumstance, as the device can then image one or more planes within the tissue that have orientations nominally parallel to the surface of the tissue being examined. Illumination system 205 then directs obliquely directs illumination light onto the tissue 290, with incidence to the tissue outside the field of view (larger numerical aperture (NA) of the objective lens. In this version of the illumination system 205, the second condenser lens 230 could be used off axis, so that it bends the light to the area being examined by detection system 210. Other means for off axis light bending, such as wedge prisms, could be used. The use of oblique or angularly off-axis illumination can be useful for the polarization diagnostic device of the present invention, as the illumination light into the tissue and imaging light coming from the tissue have less spatial overlap, and thus image contrast of the birefringent tissue structures may be higher. Note that illumination system 205 is depicted as a light source comprising multiple light emitters 225. Illumination system 205 is also depicted as including one type of light uniformization optics, an in particular an integrating bar 235, which is well known to those skilled in the art. Nominally the condenser lenses 230 would work in combination to image the output face of the integrating bar 235 to the tissue 290.

The polarization diagnostic device 200 of the present invention could potentially be yet more compact if the illumination optics and the detection optics shared a partially common optical path. A first such example is depicted in FIG. 7b (again shown without controller 215). In this case, a beam combining prism 285 is used so that illumination light from the illumination system 205 and the imaging light for detection system 210 share a common optical path between the prism 285 and the tissue 290. In particular, this means that the illumination light and the imaging light nominally traverse common optical elements (such as objective lens 240), and not the illumination and imaging light rays necessarily traverse identical ray paths through such common optical elements. Both polarization beam combining prisms and dichroic beam combining prisms could be awkward to use. In particular, the use of a polarization prism to combine imaging and illumination light in common optical path may be complicated by the fact that the polarization orientations of both the illumination and detection beams are intentionally variable. For example, this could mean that polarized illumination light could be reflected by a polarization prism to the tissue, or transmitted through it, missing the tissue, depending on the rotational orientation. On solution to this problem is to use a TIR prism for prism 285, as is shown in FIG. 7b.

In more detail, the device of FIG. 7b depicts that a light beam from light source 225 is directed off a mirror 232 and passes through condenser lens 230. In FIG. 7b, the illumination light is preferably directed to an angle-dependent beamsplitter (TIR prism 285) comprising two transparent prisms having angled surfaces internal to the overall prism 285, which are substantially parallel to each other and filled with a low refractive index material (such as air or a low index optical adhesive). The combination of the angular orientation of the internal angled surface of the first constituent prism and the refractive index of this first prism is such that the illumination light incident thereupon is reflected (at greater than the critical angle) towards tissue 290 by total internal reflection (TIR). Image light returning from tissue 290 passes through objective lens 240 and TIR prism 285 on its way to being imaged to the detector 280. This type of TIR prism has the advantage that it can be polarization insensitive. Additionally, the configuration of the constituent prisms and the internal low refractive index region is such that light returning from tissue 290 is incident upon the constituent prisms such that it is smaller than the critical angle and is totally transmitted there through. The imaging optics of the detection system can be designed so that the emerging beam image light from the tissue 290 is “on axis” with respect to the optics themselves. In the specific illustration of FIG. 7b, the collected image light in non-normally emergent from the tissue 290. But it should be understood that the various image planes in the tissue could be parallel to the tissue surface, if the detection system 210 is tilted to be normal to the tissue surface. Alternately, this problem could be addressed by tilting the detector 280. However, that creates a problem in imaging systems, known as the Scheimpflug condition, wherein the plane of best imaging is not a plane of constant magnification. The device of FIG. 7b could be designed to reside in a single housing (not shown), which would enclose both the illumination and detection optics. In that case, the overall device could then be readily held to orient the detection system 210 nominally normal to the tissue. The device 200 (any embodiment) may also include a means (likely optical or mechanical) to register or hold the device 200 in a fixed position relative to the tissue, at least during the image acquisition phase.

The device of FIG. 7b also provides a waveplate 252 after pre-polarizer 250. For example, if pre-polarizer 250 is a mini-prism type polarization converter, it may be more advantageous to rotate waveplate 252 to modify the polarization state relative to tissue 290, and leave pre-polarizer 250 fixed. Likewise, a waveplate could be rotated in the detection system instead of the polarization analyzer 255. Of course, the waveplate in these cases is likely an extra component, so the benefit must outweigh the loss in efficiency and the additional mounting hardware and cost.

The device of FIG. 7c is another alternate embodiment for the polarization diagnostic device 200 of the present invention. Controller 215 is again not shown for simplicity, but would be provided. In this device, the light beam imaged to the detector 280 follows a light path that is nominally normal at the tissue 290, which means that the Scheimpflug condition is avoided. To enable this, illumination system 205 and detection system 210 are combined to share a common optical path with respect to the tissue 290 by use of a polarization beamsplitter 280, which is shown as comprising two right angle prisms. Objective lens 240 then handles both the illumination and imaging beams. For example, this polarization beamsplitter 287 could be a MacNeille type prism or a wire grid polarizer assembled onto a prism substrate. As before, the polarization states provided by illumination system 205 and collected by detection system 210 are again nominally orthogonal to each other. However, in this case, the pre-polarizer and the polarization analyzer are nominally held in fixed orthogonal orientations, while these orthogonal polarization states are rotated simultaneously by waveplate 252. This same approach can also be used for the device of FIG. 7b, by placing a waveplate between prism 285 and lens 240. For either device, this has the advantage that a single motor (nominally a stepper motor) is needed to rotate polarizations, eliminating parts and a motor synchronization issue. On the other hand, for the FIG. 7c device, this means the portion of the light returning from tissue 290 that is of the polarization state that is not transmitted by the polarization beamsplitter (PBS) 287 towards detector 280, is then reflected by polarization beamsplitter 287 back towards the light source. Care then may be needed to minimize stray reflections that could ultimately affect image contrast.

The device of FIG. 7c is also illustrative of other design options for polarization diagnostic device 200. For example, the light source is again depicted as an array of light emitters 225. However, in this case, there is space in the middle, such that there are no on-axis light emitters. If viewed in three dimensions, light emitters 225 could be thus configured as a ring or annular light source. In this case, the illumination light could be provided to the tissue 290 at angles larger than the imaging collection angle (relative to objective lens 240). In particular, the smallest numerical aperture (NA) of the illumination light is larger than the NA of the collected and imaged light. This type of off axis illumination could be advantageous in enhancing the dynamic range of the detection system, as the illumination light and image light will have less overlap in the optics (waveplate 252 and lens 240) and in the tissue 290.

The device of FIG. 7c also shows the detection system 210 as comprising a polarization analyzer having multiple components, such as a polarization analyzer 255 and a polarization beamsplitter 260 tilted at a nominal angle of 45 degrees. This device configuration can provide improved polarization extinction over the earlier configurations in which the polarization analyzer 255 could comprise two near parallel polarizers, as the light rejected by polarization beamsplitter 260 is nominally transmitted straight through and exits the system (and can be trapped in a beam dump). To further provide enhanced dynamic range, both polarization analyzer 255 and polarization beamsplitter 260 are preferably both wire grid polarizers. Preferentially, for highest contrast, the wires 410 of the wire grid polarization beamsplitter 260 are on the side of the plate substrate 420 that is closest to detector 280, rather than the further surface. Polarization contrast might be further improved if a polarization compensator (not shown) was used and/or if the wire grid polarization beamsplitter is rotated in plane, as was described respectively in U.S. Patent Application Publication No. 2003/0128320 (Mi et al.), and U.S. Pat. No. 6,805,445 (Silverstein et al.), both of which are commonly-assigned to the same assignee as the present invention. The detection subsystem nominally comprises PBS 260, analyzer 255, lens 242, field lens 245 and detector 280. Any focusing optics (such as lens 242) could also be reflective, instead of refractive. Although the detection subsystem, including detector 280, could be variably rotated to look at the collagen network, it is much easier to rotate a waveplate 252. In the case that polarization beamsplitter 287 is also a plate type (rather than a cube) polarizer, for example identical to the wire grid plate polarization beamsplitter 260, then it could be advantageous to switch the positioning of illumination system 205 and detection system 210. That is, illumination light would be transmitted through the polarization beamsplitter 287 to tissue 290, while imaging light would emerge from tissue 290 and be reflected off the plate PBS 287 and into detection system 210. With these changes, polarization beamsplitter 287 can be a plate type polarizer (like the wire grid polarizer depicted in FIG. 8) while avoiding the well-known astigmatism problems that occur with having an imaging beam transit a tilted plate.

It is noted that waveplate 252 is nominally a quarter wave plate, although other retardances (such as λ/8 retardance) could be used. It is also noted that a similar effect might be obtained by rotating the entire device 200 relative to the tissue 290. Thus, the fixed polarization states could be rotated relative to the tissue without the need for waveplate 252. However, in that case, the orientation of detector 280 would also change relative to the tissue, which would prevent a consistent set of images from being obtained. Moreover, rotation of waveplate 252 is easy, as it enables the controlled rotation of a low weight mechanical mass.

In the prior discussions, it has been suggested that images can be obtained at various depths into the tissue by having a light source provide wavelength sequential output, where the variable tissue absorption and light penetration with wavelength, along with image processing by controller 215, is used to provide images at different depths. Of course, as the wavelengths are varied, the plane of best focus in the tissue will change as well. The image quality provided by polarization diagnostic device 200 might be improved if the device is equipped with a best focus adjustment, such as an auto-focus or zoom capability. As an example, the device of FIG. 7c is depicted with an arrow adjacent to objective lens 240 to indicate the potential for a variable focus adjustment. The motion of objective lens 240 would nominally be controlled by a mechanism (not shown) and controller 215. The use of a variable focus may improve the dynamic range (signal to noise) of the device. Variable focus could also allow the device to be simplified while obtaining good image quality with tissue depth, as the light source may need to provide fewer wavelengths (for example, maybe just 630 nm and 830 nm) or even just one wavelength for tissue examination. Also, while the controller 215 would need variable focus control capabilities, it might need less software and image processing algorithms to provide quality imagery of the collagen network at different tissue depths.

As another note, the devices of FIGS. 7b and 7c both utilize a common lens (objective lens 240), which directs the illumination light to the tissue 290 and collects the image light from the tissue 290. As objective lens 240 is moved through focus, it will not only influence the focal planes that are imaged by detection system 210, but the distribution of the illumination light as well. In that context, it should be understood that the illumination system 205 would preferably be designed so that the condensing lenses 230 and the objective lens 240 work together such that the illumination light will nominally illuminate a larger area than the image area examined by detection system 210 for all focal positions of objective lens 240.

The polarization diagnostic device 200 of FIGS. 7b and 7c may also be well equipped to be a light therapy device. In this case controller 215 would drive the light source (shown as light emitters 225) to provide the desired light dosage. In light therapy mode, controller 215 could potentially control the wavelengths, intensities, dosage times, and modulation frequencies of the light emitted from the light source, such as to provide wavelength sequential illumination. As such, the device could provide sequential multi-spectral illumination (for example, red followed by IR), or simultaneous multi-spectral illumination (for example, red and IR). The illumination system 205 would then produce polarized light, which would traverse polarization beamsplitter 287 and objective lens 240 and illuminate the tissue 290. Alternately, the objective lens 240 could be removed, so that a larger area of tissue is illuminated with therapeutic light.

Of course, polarization diagnostic device 200 could be used in a tissue diagnostic mode in conjunction with a separate light therapy device (300) to treat tissue (for example pressure ulcer 170) as depicted in FIG. 9. However, a single device that is capable of both tissue diagnostic and light therapy functions could reduce the burden on the clinician, as well as have cost advantages. The device of the present invention (in particular, the devices of FIGS. 7a-7c) could be first used to assess the condition of the tissue by providing digital images and diagnostic metrics. A clinician could then use this information to determine a light therapy treatment protocol, in terms of the light dosage to be applied. For the instances in which device 200 of the present invention is used for light therapy, the detection system 210 may be temporarily disabled during light therapy operation. Alternately, the detection system 210 could be used in diagnostic mode, not only before light therapy, but also during and after light therapy. The device of FIG. 7b may be best suited for dual use, as the illumination and detection polarization states could potentially be independently controlled, allowing the device to look for different characteristics of the tissue or light application. In any case, any of the FIGS. 7a-7c devices could be used prior to light therapy to examine the collagen network and the healing ECM. These devices could be used during light therapy to examine any effects on the fibroblasts, collagen networks, or other aspects of the ECM. However, as there is typically a time delay of hours or days between therapeutic light application and affects on some types of cellular activity (for example; angiogenesis), the detection system 210 might be better used to examine effects that have shorter time constants.

As another point, the device of the present invention might be used to detect and image tissues laden with bio-chemicals associated with wound healing. For example, light source 225 could be driven to illuminate the tissue with light of some pre-determined wavelength. To enable this, light source 225 might provide either UV or blue light, which is generally known to be useful in stimulating fluorescence. This light could then be absorbed and induce fluorescence or chemi-luminescence from a bio-chemical in the tissue, which could then be detected by the detection system 210. Alternately, any natural (not induced) light emissions could likewise potentially be observed. The high dynamic range or the optical system of the present invention could enable such detection. The detection system 210 could also be equipped with a tunable spectral filter, such as the previously discussed liquid crystal tunable filter, so that the detection system images the light emitted by the tissue bio-chemicals of interest, while excluding light from other sources. With respect to the devices of FIG. 7b or 7c, the tunable spectral filter would preferentially be placed in the respective detection system 210, somewhere between the beamsplitter 285 and the detector 280. As examples, the device could be used to look for bio-chemical marker concentrations of actin, hydroxylproline nitrates, or NADH or MMPs (matrix metallo-proteinases), which are associated with various aspects of healing progress or inhibition (including infection).

Throughout the prior discussion, it has been assumed that the pre-polarizer and the polarization analyzer are orthogonally oriented or crossed, and the resulting extinction axes are iteratively rotated in tandem relative to the tissue 290. Alternately, for improved imaging of the birefringent tissue structures, it may be desirable to independently and interactively rotate each of the two polarizers, without maintaining the synchronicity of motion. For example, pre-polarizer 250 could be rotated from some initial angular position, through N steps to some final angular position. Then, at each of the N positions, polarization analyzer 255 could be rotated through M steps. For example, pre-polarizer could start at a first position (0°), and then rotate sequentially to 30°, 60° and 90° (N=3). At each of these positions, polarization analyzer 255 could rotate from 0° (aligned to the pre-polarizer) to 30°, 60°, and 90° (crossed with the pre-polarizer) for M=3 steps and four positions. As can be seen, this approach allows a more variable range of polarization states to be presented to the tissue and then examined, but likewise, more data must be collected and analyzed. However, this approach allows the detector to examine light that re-emerges from the tissue with polarization states near those of the incident illumination light. In some cases (provided that a specular reflection of the first tissue surface is eliminated), this may improve the image quality. The devices of FIGS. 7a and 7b could operate in this mode, but the device of FIG. 7c could not, as it assumes the illumination and imaging polarization states are fixed relative to polarization beamsplitter 287.

A few further comments regarding the manner of use of the present invention are worthwhile. In practice, the clinician could see the patient, remove whatever bandages may be present, and inspect the wounds. As part of this process, the clinician could examine the wounded tissue using the polarization diagnostic device 200 the present invention. The polarization diagnostic device of the present invention could enable the clinician to ascertain various properties of the collagen network (collagen fiber size and length, fiber orientation, fiber density, collagen type (I or III, for example), collagen mesh structure with tissue depth, etc.). The clinician could then make an assessment of the tensile strength of the collagen structures. The clinician could also use this device to examine multiple areas of the wounds that may exhibit different states of healing, while also using the device to examine adjacent normal tissues for comparison. As the present device is mainly intended to look at wound healing in the skin, and in particular the collagen network in the skin, the clinician may use complementary methods to aid the diagnostic process. Recall that the collagen network in skin is irregular, having generally a local pre-dominant direction, but also sufficient multi-directionality to respond to stretching from any direction. The clinician could take advantage of this, by applying mild pressure adjacent to the wound, and using polarization diagnostic device 200 to examine the mechanical stress response of the collagen networks of the normal skin and the healing skin, to better understand the condition of the rebuilding ECM.

The clinician could also use the device of the present invention to assess the progress of angiogenesis in wound healing. In particular, by examining the extent, density, and size of the capillaries, a clinician could then understand whether the tissue is obtaining sufficient blood flow to progress. It should also be understood that the device of the present invention might be used to examine other birefringent tissue structures, such as tendons, ligaments, and muscles.

Once the clinician has used the polarization diagnostic device 200 of the present invention, and therefore understands the conditions of the ECM in and around the wound, the clinician could use this information in a variety of ways to improve the patient care. For example, the clinician could determine that collagen forming in the ECM lacks sufficient structural integrity (relative to bundle length, diameter, density, orientation) for proper granulation, and then that the fibroblasts need directed stimulation. As one example, the light therapy technique of U.S. Pat. No. 6,676,655 (McDaniel), which employs pulsed femtosecond yellow laser light (590 nm) to induce stimulatory effects in fibroblasts, could be employed. More generically, light therapy devices generally, including polarization diagnostic device 200, could be used to provide therapeutic light to the tissues. The clinician could also use the information to decide to employ topical agents or growth factors that impact fibroblasts, or other processes such as angiogenesis, epithelialization, or granulation. The clinician could also use this information as a guide in the use of collagen matrix wound care products (such as the Matrix Collagen Sponge Wound Dressing from Collagen Matrix Inc., Franklin Lakes, N.J.), skin grafts (such as Apligraf from Organogenesis, Canton Mass.), or various bandages (hydrocolloidal, alginates, silver based, etc.).

The discussion of the present invention has been primarily focused on enabling tissue state assessment for wound healing. However, it should be understood that the device of the present invention could potentially be used for other medical applications. For example, photo-damage, such as from over exposure to sunlight, is known to cause cancerous conditions such as melanomas. As an aspect of this, photo-damage also causes collagen thinning in skin. Therefore, the present device might be used to examine the collagen structures in skin as screening method for assessing potentially pre-cancerous conditions. Similarly, the device could be used to examine the tensile strength of scars, to look for the potential of skin breakdown, and thus potentially enable a clinician to prevent scar deterioration. As another example, burn scar tissue tends to heal in a manner that restricts patient motion, which can be later corrected by cosmetic surgery by plastic surgeons. This device could provide a plastic surgeon with valuable information regarding the collagen structures within the scar, such that the surgery could be better directed. Likewise, a dermatologist or cosmetic surgeon could use this device to assess the collagen structures underneath fine lines and wrinkles, as a guide to treatment.

The invention has been described in detail with particular reference to a presently preferred embodiment, but it will be understood that variations and modifications can be effected within the scope of the invention. The presently disclosed embodiments are therefore considered in all respects to be illustrative and not restrictive. The scope of the invention is indicated by the appended claims, and all changes that come within the meaning and range of equivalents thereof are intended to be embraced therein.

### Parts List

100 skin
105 epidermis
110 dead epithelial cells
115 basement membrane (basal lamina)
120 reticular dermis
125 blood capillary
127 red blood cells
130 proteoglycans
140 fibroblasts
145 collagen fiber bundles
150 collagen fibrils
160 human body
165 Langer's cleavage lines
170 pressure ulcer
200 polarization diagnostic device
205 illumination system
210 detection system
215 controller
220 light source
222 filters
225 light emitters
230 condenser lens
232 mirror
235 integrating bar
240 objective lens
242 lens
245 field lens
250 pre-polarizer
252 waveplate
255 polarization analyzer
260 polarization beamsplitter
270 illumination optical axis
275 imaging optical axis
280 detector
285 TIR prism
287 polarization beamsplitter
290 tissue sample
300 light therapy device
400 wire grid polarizer
410 wires
420 dielectric substrate
430 beam of light
440 reflected light beam
450 transmitted light beam

1. A polarization based diagnostic device for optically examining the medical condition of tissue comprising:
a) an illumination optical system, comprising a light source, having one or more light emitters, and beam shaping optics, which together provide illumination light to the tissue being examined;
b) an optical detection system, comprising imaging optics and an optical detector array, for detecting light from said tissue;
c) polarizing optics, provided in both said illumination optical system and said optical detection system, which are crossed so as to pass orthogonal polarization states;
d) wherein iterative rotational means are provided to rotate the orthogonal polarization states relative to the tissue being examined;
e) image enhancement means, including some combination of image processing, sequential multi-spectral illumination and imaging, and image focus control to facilitate quality imaging at varying depths within said tissue; and
f) a controller for operating said light source, said detector array, said sequential multi-spectral illumination and imaging, and said image focus control, as well as providing image processing of the captured images to aid the diagnostic process.

2. A device as in claim 1 wherein said device is used to examines birefringent tissue structures in skin tissues.

3. A device as in claim 1 wherein said device is used to examine a collagen network in said tissue.

4. A device as in claim 1 wherein said device is used to examine granulation tissue in wound healing, including a status of collagen formation and/or angiogenesis.

5. A device as in claim 1 wherein said device is used to examine birefringent tissue structures in an extra cellular matrix.

6. A device as in claim 1 wherein said polarizing optics comprise wire grid polarizers.

7. A device as in claim 1 wherein said the polarizing optics includes a polarization converter.

8. A device as in claim 1 wherein said orthogonal polarization states are rotated by rotating the polarizing optics in the illumination system synchronously with rotating polarizing optics in the detection system.

9. A device as in claim 1 wherein said light source comprises one or more LEDs, laser diodes, or super-luminescent diodes.

10. A device as in claim 1 wherein said light source provides light to the tissue within a spectral range of 500-1300 nm.

11. A device as in claim 1 wherein said illumination system includes a tunable spectral filter.

12. A device as in claim 1 wherein said beam shaping optics include light uniformization means, such as Kohler illumination, an integrating bar, or a fly's eye integrator.

13. A device as in claim 1 wherein said illumination system provides incident light to the tissue at angles larger than those collected by the detection system.

14. A device as in claim 1 wherein said illumination system illuminates an area on the tissue larger than that imaged by the detection system.

15. A device as in claim 1 wherein said illumination system and detection system partially share a common optical path, with at least a beamsplitter and an objective lens being used in common.

16. A device as in claim 1, wherein said illumination light in incident onto said tissue as off-axis light.

17. A device as in claim 1 wherein said beamsplitter is an angle sensitive TIR prism.

18. A device as in claim 1 wherein said beamsplitter is a PBS.

19. A device as in claim 1 wherein said imaging optics comprise an objective lens.

20. A device as in claim 19 wherein said objective lens is positioned by said focus control.

21. A device as in claim 7 wherein said detector array is a CCD or CMOS sensor.

22. A device as in claim 7 wherein said detection system includes a tunable spectral filter.

23. A device as in claim 7 wherein said image processing includes the operation of applying corrections from images collected at shallow tissue depths to improve the image quality of images collected from deeper tissue depths.

24. A device as in claim 1 wherein said device is used to examine wounds of various sorts, and the progress in the healing thereof.

25. A device as in claim 1 wherein said device is used to examine the structure and integrity of the collagen network in an extra-cellular matrix.

26. A device as in claim 1 wherein said device is used to both examine said tissue and adjacent tissue for comparison.

27. A device as in claim 1 wherein said device is used to examine tissue while applying pressure to adjacent tissue so as to examine the structure of said tissue while under mechanical stress.

28. A device as in claim 1 wherein said device and the information obtained there-from is used to guide further treatment efforts to heal said tissue.

29. A device as in claim 1 wherein said device can also be operated as a light therapy device.

30. A device as in claim 1 wherein said device is used in conjunction with a light therapy device.

31. A polarization based diagnostic device for optically examining the medical condition of tissue, comprising:
a) an illumination optical system, comprising a light source, having one or more light emitters, illumination beam shaping optics, and polarizing optics, for providing illumination light;
b) an optical detection system, comprising focusing optics, an optical detector array, and polarizing optics which are nominally wire grid polarizers, which receives image light;
c) a polarization beamsplitter for re-directing either said illumination light or said image light such that said illumination light and said image light traverse a common optical path between said polarization beamsplitter and said tissue;
d) a waveplate, located after the beam combining optics, for simultaneously rotating the polarized illumination and polarized imaging light;
e) an objective lens with a focus control means for directing illumination light to the tissue and collecting image light from the tissue;
f) a controller for operating the light source, the detector array, the focus control, the rotational motion of the waveplate, and for providing image processing of the captured images to aid the diagnostic process;
g) an image enhancement means within said controller; and
wherein said polarizing optics, provided in both said illumination optical system and said optical detection system, are crossed so as to comprise orthogonal polarization states.

32. A device as in claim 31 wherein said light source can provide sequential multi-spectral illumination, either by direct operation of said light source, or by direct operation of said light source and a tunable spectral filter.

33. A device as in claim 31 wherein said polarizing optics in said detection system include a wire grid polarization beam splitter and a wire grid polarizer.

34. A device as in claim 31 wherein said polarization beamsplitter can either be wire grid polarization beam splitter or a MacNeille type polarization beamsplitter.

35. A device as in claim 31 wherein said device is used to examine the birefringent tissue structures in skin, including an extra-cellular matrix of said skin.

36. A device as in claim 31 wherein said device is used to examine a collagen network in said tissue.

37. A device as in claim 31 wherein said polarizing optics in said illumination system include a polarization converter.

38. A device as in claim 31 wherein said waveplate is rotated iteratively through some angular range, such as 90 degrees.

39. A device as in claim 31 wherein said light source comprises one or more LEDs, laser diodes, or super-luminescent diodes.

40. A device as in claim 31 wherein said device can also be operated as a light therapy device.

41. A device as in claim 31 wherein said illumination system provides incident light to said tissue at angles larger than those collected by said detection system.

42. A device as in claim 31 wherein said illumination system illuminates an area on said tissue larger than that imaged by said detection system.

43. A device as in claim 31 wherein said detection system can include a tunable spectral filter.

44. A device as in claim 31 wherein said image enhancement means uses image processing to improve the image quality of images collected from deeper in said tissue, by applying corrections from images collected at shallower depths in said tissue.

45. A device as in claim 31 wherein said image enhancement means includes which some combination of image processing, sequential multi-spectral illumination and imaging and image focus control to facilitate quality imaging at varying depths within said tissue.

46. A polarization based diagnostic device for optically examining the medical condition of tissue, comprising:
a) an illumination optical system, comprising a light source, having one or more light emitters, illumination beam shaping optics, and polarizing optics, for providing illumination light to said tissue;
b) an optical detection system, comprising imaging optics, an optical detector array, and polarizing optics which are nominally wire grid polarizers, which receives image light from said tissue;
c) a beamsplitter for re-directing either said illumination light or said image light such that said illumination light and said image light traverse a common optical path between said beamsplitter and said tissue;
d) a waveplate, located between said beamsplitter and said tissue, for simultaneously rotating the polarized illumination and polarized imaging light;
e) an objective lens with a focus control for directing said illumination light to said tissue and collecting said image light from said tissue;
f) a controller for operating at least said light source, said detector array, said focus control, said rotational motion of said waveplate, and for providing image processing of the captured images to aid the diagnostic process;
wherein said polarizing optics, provided in said illumination optical system and said optical detection system, are crossed so as to define nominally orthogonal polarization states; and
wherein said illumination light is controlled to provide a sequential multi-spectral illumination of said tissue.

47. A polarization based diagnostic device for optically examining the medical condition of tissue, comprising:
a) an illumination optical system, comprising a light source, having one or more light emitters, illumination beam shaping optics, and polarizing optics, for providing illumination light to said tissue;
b) an optical detection system, comprising imaging optics, an optical detector array, and polarizing optics which are nominally wire grid polarizers, which receives image light from said tissue;
c) a beamsplitter for re-directing either said illumination light or said image light such that said illumination light and said image light traverse a common optical path between said beamsplitter and said tissue;
d) an objective lens with a focus control for directing said illumination light to said tissue and collecting said image light from said tissue;
wherein said polarizing optics, provided in said illumination optical system and said optical detection system, are independently and iteratively rotated so as to define variable polarization states relative to said tissue;
wherein said illumination light is controlled to provide a sequential multi-spectral illumination of said tissue; and
e) a controller for operating at least said light source, said detector array, said focus control, said rotational motion of said polarizers, and for providing image processing of the captured images to aid the diagnostic process.

48. A polarization based diagnostic device for optically examining the medical condition of tissue, comprising:
a) an illumination optical system, comprising a light source, having one or more light emitters, illumination beam shaping optics, and polarizing optics, for providing illumination light to said tissue;
b) an optical detection system, comprising imaging optics, polarizing optics, and an optical detector array, which receive image light from said tissue;
c) a beamsplitter for re-directing either said illumination light or said image light such that said illumination light and said image light traverse a common optical path between said beamsplitter and said tissue;
d) an objective lens with a focus control for directing said illumination light to said tissue and collecting said image light from said tissue;
wherein said polarizing optics, provided in said illumination optical system and said optical detection system, are crossed so as to define nominally orthogonal polarization states;
wherein said polarizing optics in said illumination optical system and said optical detection system are rotated iteratively, in unison, relative to the tissue being examined; and
e) a controller for operating at least said light source, said detector array, said focus control, said rotational motion, and for providing image processing of the captured images to aid the diagnostic process.

49. A device as in claim 48 wherein said illumination light is controlled to provide a sequential multi-spectral illumination of said tissue.

50. A device for light application to tissue in vivo, having a diagnostic operational mode which examines the condition of the said tissue by using polarized light, and having a therapeutic operational mode, which applies polarized incident light to said tissue, comprising:
a) an illumination optical system, comprising a light source, having one or more light emitters, illumination beam shaping optics, and polarizing optics, for providing illumination light;
b) an optical detection system, comprising focusing optics, polarizing optics, and an optical detector array which receives image light;
c) a beamsplitter for re-directing either said illumination light or said image light such that said illumination light and said image light traverse a common optical path between said beamsplitter and said tissue;
d) an objective lens with a focus control means for directing illumination light to said tissue and collecting image light from said tissue;
wherein said polarizing optics, provided in said illumination optical system and said optical detection system, are independently and iteratively rotated so as to define variable polarization states relative to said tissue;
wherein said illumination light is controlled to provide multi-spectral illumination of said tissue; and
e) a controller, used in both diagnostic mode and therapy mode, for operating said light source, said detector array, said focus control, and said rotational motion of said polarizers.

51. An apparatus for light application to tissue in vivo, having a diagnostic light operational mode which examines the condition of the said tissue by using polarized light, and having a therapeutic light operational mode, comprising:
a) an illumination optical system, comprising a light source, having one or more light emitters, illumination beam shaping optics, and polarizing optics, for providing illumination light;
b) an optical detection system, comprising focusing optics, and polarizing optics, an optical detector array, which receives image light;
c) beam combining optics comprising a polarization beamsplitter for combining the illumination light and the imaging light;
d) a waveplate, located after the beam combining optics, for simultaneously rotating the polarized illumination and polarized imaging light;
e) an objective lens with a focus control means for directing illumination light to the tissue and collecting image light from the tissue;
wherein the polarizing optics, provided in both said illumination optical system and said optical detection system, are crossed so as to comprise orthogonal polarization states; and
f) a controller, used in both diagnostic mode and therapy mode, for operating said light source, said detector array, said focus control, said rotational motion of said waveplate.

52. A polarization based diagnostic device for optically examining the medical condition of tissue, comprising:
a) an illumination optical system, comprising a light source, which provides illumination light to said tissue;
b) an optical detection system, comprising imaging optics and an optical detector array;
c) a first polarizer in said illumination system;
d) a second polarizer in said detection system;
e) wherein said first polarizer and said second polarizer are crossed so as to pass orthogonal polarization states; and
f) wherein said first and second polarizers are rotated iteratively, in unison, relative to the tissue being examined.

53. A polarization diagnostic device as in claim 51 wherein said device includes a controller which operates said light source, said detection system, a multi-spectral illumination system, a focus control, rotation of said polarizers, and an image analysis system.

54. A polarization diagnostic device as in claim 52 wherein said device include an image enhancement means selected from a group comprising image processing, sequential multi-spectral illumination, and image focus control for imaging at varying depths within said tissue.

55. A polarization based diagnostic device for optically examining medical condition of tissue, comprising:
a) an illumination optical system for providing sequential multispectral illumination to said tissue;
b) an optical detection system for detecting and focusing light from said tissue;
c) a first polarizer in said illumination system;
d) a second polarizer in said detection system; and
e) wherein said first and second polarizers are independently and iteratively rotated so as to define variable polarization states relative to said tissue.

56. A polarization diagnostic device as in claim 55 wherein said device includes a controller which operates said light source, said detection system, a multi-spectral illumination system, a focus control, rotation of said polarizers, and an image analysis system.

57. A polarization diagnostic device as in claim 55 wherein said device include an image enhancement means comprising some combination of image processing, sequential multi-spectral illumination, and image focus control for imaging at varying depths within said tissue.

58. A polarization based diagnostic device for optically examining the medical condition of tissue, comprising:
a) an illumination optical system comprising a light source, which provides illumination light to said tissue;
b) a first polarizer in said illumination system;
c) an optical detection system comprising imaging optics and an optical detector array for detecting image light from said tissue;
d) a second polarizer in said detection system;
e) a beamsplitter for re-directing either said illumination light or said image light such that said illumination light and said image light traverse a common optical path between said beamsplitter and said tissue;
f) wherein said first polarizer and said second polarizer are crossed so as to define nominally orthogonal polarization states; and
g) a wave plate, located between said beamsplitter and said tissue, wherein said wave plate is rotated iteratively.

59. A polarization diagnostic device as in claim 58 wherein said device includes a controller which operates said light source, said detection system, a multi-spectral illumination system, a focus control, rotation of said polarizers, and an image analysis system.

60. A polarization diagnostic device as in claim 58 wherein said device include an image enhancement means comprising some combination of image processing, sequential multi-spectral illumination, and image focus control for imaging at varying depths within said tissue.

61. A polarization based diagnostic device for optically examining the medical condition of tissue, comprising:
a) an illumination optical system, comprising a light source, having one or more light emitters and illumination beam shaping optics for providing illumination light;
b) an optical detection system, comprising focusing optics and an optical detector array which receives image light;
c) polarizing optics, provided in both said illumination optical system and said optical detection system, which are crossed so as to pass orthogonal polarization states;
wherein iterative rotational means are provided to rotate the polarizers and thus rotate the orthogonal polarization states relative to the tissue being examined;
d) beam combining optics for combining the illumination light and the imaging light;
e) an objective lens with a focus control means for directing illumination light to the tissue and collecting image light from the tissue;
f) a controller for operating the light source, the detector array, the focus control, the rotational motion of the polarizers, and for providing image processing of the captured images to aid the diagnostic process; and
g) image enhancement means within said controller, which utilizes some combination of image processing, sequential multi-spectral illumination and imaging and image focus control to facilitate quality imaging at varying depths within said tissue.

62. A method for using a polarization based diagnostic device to examine the condition of tissue comprising:
a) directing polarized illumination light at the tissue using an illumination system;
b) collecting image light which is of an orthogonal polarization state to said illumination light from said tissue with an imaging system;
c) iteratively and synchronously rotating the polarization state of the illumination and the orthogonal polarization state collected by said imaging system;
d) processing image data collected by said imaging system to produce digital images and metrics related to the state of the tissue;
e) refocusing said imaging system; and
f) collecting additional images at different depths in the tissue.

63. A method as in claim 62 wherein said illumination light is varied to provide sequential multi-spectral illumination.

64. A method as in claim 62 wherein the tissue being examined is compared to other adjacent tissue by using said device.

65. A method as in claim 62 wherein tissue adjacent to the tissue being examined is subject to mechanical pressure, so to mechanically stress the tissue being examined with said device.

66. A method for using a polarization based diagnostic device to examine the condition of tissue in conjunction with light therapy, including:
a) directing polarized light at the tissue using an illumination system;
b) collecting polarized image light emerging from said tissue with an imaging system;
c) detecting said image light to produce image data;
d) processing said image data to produce digital images and diagnostic metrics concerning the state of the tissue;
e) applying the image data and diagnostic metric data to determine a protocol for light therapy treatment; and
f) applying light therapy as a treatment.

67. A method as in claim 66 wherein images are collected at different depths in the tissue, by using focus control, multi-spectral illumination and imaging, and image processing in some combination to obtain quality images.

68. A method for light therapy, comprising application of light to a patient in combination with diagnosis of the condition of the collagen network within the tissue being treated, the method comprising:
a) applying a therapeutic light treatment method to a patient;
b) sensing the condition of the collagen network in the tissue of said patient, by detecting the effect the birefringence of said collagen network has on an incident diagnostic light beam;
c) assessing the need for applying said therapeutic light treatment onto said tissue, and thereby optimizing the dosage of the light therapy relative to various parameters, including the wavelength, intensity, modulation, repeat frequency, and location of light application onto said tissue; and
d) modifying the dosage parameters of said therapeutic light treatment in accordance with the detected condition of said collagen network.

69. A polarization based diagnostic device for optically examining the medical condition of tissue, comprising:
a) an illumination optical system, comprising a light source, having one or more light emitters, illumination beam shaping optics, and polarizing optics, for providing illumination light to said tissue;
b) an optical detection system, comprising imaging optics, an optical detector array, and polarizing optics which are nominally wire grid polarizers, which receives image light from said tissue;
c) a beamsplitter for re-directing either said illumination light or said image light such that said illumination light and said image light traverse a common optical path between said beamsplitter and said tissue;
d) an objective lens with a focus control for directing said illumination light to said tissue and collecting said image light from said tissue;
wherein said illumination light is controlled to provide a sequential multi-spectral illumination of said tissue;
e) a controller for operating at least said light source, said detector array, said focus control, and for providing image processing of the captured images to aid the diagnostic process; and
wherein said device include an image enhancement means selected from a group comprising image processing, sequential multi-spectral illumination, and image focus control for imaging at varying depths within said tissue.
